(19) United States
(12) Patent Application Publication
(10) Pub. No.: US 2026/0115312 A1
(43) Pub. Date: Apr. 30, 2026

(54) Conjugates for neuroretinal drug delivery

(71) Applicant: Mireca Medicines GmbH, Tübingen (DE)

(72) Inventors: Gustav Christensen, Tübingen (DE); François Paquet-Durand, Tübingen (DE)

(73) Assignee: Mireca Medicines GmbH, Tübingen (DE)

(21) Appl. No.: 19/117,094

(22) PCT Filed: Oct. 6, 2023
(86) PCT No.: PCT/EP2023/077664

§ 371 Date: Mar. 31, 2025

(30) Foreign Application Priority Data
Oct. 6, 2022 (EP) 22200008.5

(51) Int. Cl. A61K 47/69; A61K 31/7068; A61K 47/54; A61P 27/02

(52) U.S. Cl. CPC A61K 47/6911; A61K 31/7068; A61K 47/542; A61P 27/02

(57) ABSTRACT

The present invention is in the field of drug delivery. The invention relates to the use of monocarboxylates as ligands for receptors that are present in for instance the retina. These receptors were found to be able to mediate uptake of these ligands, and of conjugates of these ligands. Such conjugates can be used in methods for treating or preventing neurodegenerative diseases, such as retinal degeneration.

### FIELD OF THE INVENTION

The present invention is in the field of drug delivery. The invention relates to the use of monocarboxylates as ligands for receptors that are present in for instance the retina. These receptors were found to be able to mediate uptake of these ligands, and of conjugates of these ligands. Such conjugates can be used in methods for treating or preventing neurodegenerative diseases, such as retinal degeneration.

### BACKGROUND OF THE INVENTION

There is a paucity of drug treatments available for patients suffering from retinal diseases. An important reason for this is the lack of suitable delivery systems that can achieve sufficiently high drug uptake in the retina and its photoreceptors.

Inherited retinal degeneration (IRD) relates to a group of diseases, including retinitis pigmentosa and Leber's congenital amaurosis, characterized by the progressive loss of photoreceptors, which ultimately leads to blindness. Typically, IRD-type diseases display a primary loss of rod photoreceptors, which are responsible for vision under dim light conditions. Accordingly, initial disease symptoms include night-blindness. Once rods are lost, the cone photoreceptors, which mediate color and high acuity vision under daylight conditions, are also degenerating, ultimately leading to complete blindness. To date, IRD-type diseases remain essentially untreatable, creating a high need for new therapeutic developments.

In many types of IRD, high levels of cyclic guanosine monophosphate (cGMP) in rod photoreceptors, caused for instance by the impairment of phosphodiesterase 6 (PDE6), are found. PDE6 regulates cGMP levels and dysregulation can cause cGMP to reach pathological concentrations and over-activate important cGMP-dependent proteins, eventually leading to cell death. To mitigate this effect, drug candidates, which are inhibitory analogues to cGMP, have been shown to promote survival of photoreceptors in mouse models of IRD (Vighi, E., et al., PNAS, 2018, 115(13):E2997-E3006). Rescuing rod photoreceptors can provide functional protection of cone photoreceptors

To ensure a successful translation for clinical use, the drugs such as cGMP analogues would need to be delivered to photoreceptors in high enough concentrations to provide a protective effect. For this, a suitable drug delivery system is necessary. Glutathione-conjugated liposomes were previously shown to enhance the therapeutic effect of CN03 in IRD mouse models after systemic administration (Vighi et al., see above). These liposomes were designed for drug permeation across the blood-retinal barrier. However, intravitreal (IVT) administration is preferable to achieve high drug concentrations at the target and to limit systemic exposure. There is a need for a method of administration that is capable of directly targeting photoreceptors to obtain a therapeutic effect.

Monocarboxylate transporters (MCTs) are membrane-bound transporters highly expressed in tissues which have a substantial energy demand and thus a high turnover rate of metabolites, including the retina (Halestrap, A. P. and M. C. Wilson, IUBMB Life, 2012. 64(2): p. 109-19). MCTs are highly expressed on photoreceptors, possibly due to the demand for lactate shuttling between photoreceptors and Müller glial cells. MCTs have a variety of substrates, including lactate, pyruvate, ketone bodies, and short-chain fatty acids. MCTs have been used as a means of targeting drug delivery to the brain (Venishetty et al., doi: 10.1016/j.nano.2012.08.004).

There is a need for delivery strategies that target the retina or photoreceptors. There is a need for substances and compositions that can be used for targeting the retina or photoreceptors.

### SUMMARY OF THE INVENTION

The inventors identified strong lactate transporter (aka monocarboxylate transporter, MCT) expression on photoreceptors as a target for drug delivery vehicles. Nanoparticles were conjugated with different monocarboxylates as ligands, including lactate, pyruvate, and cysteine. For instance, monocarboxylate conjugated dye-loaded liposomes were tested on both human-derived cell-lines and on murine retinal explant cultures. It was found that nanoparticles conjugated with suitable ligands consistently displayed higher cell uptake than unconjugated nanoparticles. Pharmacological inhibition of MCT1 and MCT2 reduced internalization, corroborating the MCT-mediated uptake mechanism. As a particularly attractive embodiment, pyruvate-conjugated liposomes loaded with the various retinal drug substances reduced photoreceptor cell death in murine rd1 and rd10 retinal degeneration models. Notably, in retinal degeneration models, free drug solutions could not achieve the same therapeutic effect.

The invention provides a conjugate comprising a ligand for a monocarboxylate transporter (MCT); and a pharmaceutically acceptable nanoparticle. Preferably the monocarboxylate transporter is at least monocarboxylate transporter 1 (MCT1), monocarboxylate transporter 2 (MCT2), or monocarboxylate transporter 4 (MCT4). Preferably the ligand is a ligand that specifically binds to or is taken up into a retinal cell at a rate that is at least 10% enhanced as compared to control conditions selected from a) uptake in cells lacking expression of monocarboxylate transporter; b) uptake in cells pre-treated with MCT inhibitors; and c) uptake of a reference conjugate lacking a ligand for a monocarboxylate transporter; when measured at 2 hours or more after contacting the ligand with the target cell.

In some embodiments the pharmaceutically acceptable nanoparticle is a liposome, a solid lipid nanoparticle, a micelle, a carrier protein, a metal nanoparticle, a polyplex system, a lipoplex system, or a polymeric nanoparticle. Preferably the pharmaceutically acceptable nanoparticle comprises one or more phospholipids. Preferably the pharmaceutically acceptable nanoparticle comprises one or more non-cationic lipids.

In some embodiments the pharmaceutically acceptable nanoparticle further comprises a pharmaceutically active agent, preferably a neuroprotective agent, such as a photoreceptor rescuing drug, preferably a cyclic guanosine monophosphate (cGMP) analogue.

The ligand preferably comprises a free carboxylic acid moiety, preferably as comprised in a short chain fatty acid, an amino acid, or a keto acid. The pharmaceutically acceptable nanoparticle preferably comprises a water soluble polymer at its surface. It is highly preferred that the ligand is conjugated to the water soluble polymer. Preferably, wherein the ligand is of general formula (I):

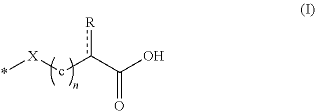

wherein X is S, O, Se, or NH; c is —CH₂—, —CH(CH₃)—, —C(═O)—, —C(═S)—, —C(═NH)—, —CH(—OH)—, —CH(NH₂)—, —CH(halogen)-, or —C(halogen)₂-; n is 1, 2, or 3; and R is —H, —CH₃, ═O, ═S, ═NH, —OH, —NH₂, or a halogen; and * is the site of conjugation to the pharmaceutically acceptable nanoparticle.

Also provided is a compound of general formula (A):

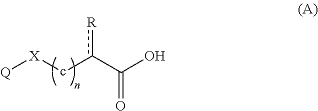

wherein X is S, O, Se, or NH; c is —CH₂—, —CH(CH₃)—, —C(═O)—, —C(═S)—, —C(═NH)—, —CH(—OH)—, —CH(NH₂)—, —CH(halogen)-, or —C(halogen)₂-; n is 1, 2, or 3; and R is —H, —CH₃, ═O, ═S, ═NH, —OH, —NH₂, or a halogen; and Q is a conjugate of a lipid and a water soluble polymer, wherein the lipid is preferably a phospholipid.

Also provided is the conjugate as defined above, for use as a medicament. Preferably the medicament is for treating a neurodegenerative disorder or a retinal disorder, such as inherited retinal degeneration (IRD), glaucoma, age-related macular degeneration, Stargardt's disease, Usher's disease, geographic atrophy, diabetic retinopathy, retinitis pigmentosa, Leber's congenital amaurosis, blindness, loss of rod photoreceptors, night-blindness, loss of cone photoreceptors, achromatopsia, loss of color vision, and loss of high acuity vision. The invention thus also provides a method of treating, delaying, or preventing a neurodegenerative disorder or a retinal disorder, the method comprising the step of administering to a subject a conjugate as defined above.

### DESCRIPTION OF THE INVENTION

It was surprisingly found that strong lactate transporter (aka monocarboxylate transporter, MCT) is expressed on photoreceptors, and the invention provides conjugates that allow the targeting of nanoparticles to MCT, and thus to photoreceptors. Accordingly the invention provides a conjugate comprising:
i) a ligand for a monocarboxylate transporter (MCT); and
ii) a pharmaceutically acceptable nanoparticle.
   Such a conjugate is referred to herein as ‘the conjugate’ or ‘a conjugate according to the invention’, as will be clear from context. The nanoparticle and the ligand form a conjugate. The ligand for an MCT is conjugated to the pharmaceutically acceptable nanoparticle, which for brevity is sometimes referred to herein as the nanoparticle.

A “conjugate” is herein defined as consisting of two entities that are coupled together. Preferably, the two entities are conjugated by covalent bonding, by non-covalent bonding, or by coordinating chemical bonding. Preferably the two entities are conjugated by covalent bonding or by non-covalent bonding. An example of non-covalent bonding is the interaction between biotin and avidin or streptavidin or another analogue. Two entities can be coupled either directly or via a (non)cleavable spacers, linkers, or other components. In preferred embodiments, the ligand for an MCT is covalently linked to a hydrophobic moiety comprised in the nanoparticle, more preferably in the lipid phase of a lipid nanoparticle such as in the lipid bilayer of a liposome. This hydrophobic moiety can be a lipid, a sterol such as cholesterol, a steroid, a vitamin or a derivative thereof such as vitamin D or a derivative thereof, vitamin E or a Vitamin E derivative (e.g. as described in EP05292820), a C8-30 alkane, a C6-30 (poly)cyclic alkane, or a C6-30 aromatic moiety. Preferably the hydrophobic moiety is a lipid or a sterol, more preferably a lipid. Preferred lipids throughout this application are phospholipids. More preferred lipids throughout this application are neutral phospholipids. In this context, a skilled artisan will appreciate that the hydrophobic moiety will phase separate into the lipid phase of the nanoparticle when it is a lipid nanoparticle. For instance, when the hydrophobic moiety is a phospholipid, it will co-assemble into the lipid bilayer of a liposome in cases where the nanoparticle is a liposome. These techniques are broadly known in the art and a skilled person will be able to select a suitable hydrophobic moiety.

In a preferred embodiment, the ligand for an MCT is linked to the hydrophobic moiety via a linker or a spacer. In preferred embodiments the ligand is solvent accessible. A preferred linker is a water soluble polymer. A water soluble polymer as defined below is conjugated at one terminus to the ligand for an MCT, and at the other terminus to the hydrophobic moiety. In preferred embodiments, the ligand for an MCT is linked to a phospholipid-PEG, as e.g. disclosed in US2015/0157733. In the most preferred embodiment, it is conjugated to 1,2-distearoyl-sn-glycero-3-phosphoethanolamine-N-[methoxy(polyethylene glycol)-2000 (DSPE-mPEG2000) or conveniently to 1,2-distearoyl-sn-glycero-3-phosphoethanolamine-N-[maleimide(polyethylene glycol)-2000]. A skilled person can select which chemistry is suitable for forming the required bonds.

A large variety of methods for conjugation of ligands with nanoparticles or lipids or polymers are known in the art. Such methods are e.g. described by Hermanson (1996, Bioconjugate Techniques, Academic Press), in U.S. Pat. Nos. 6,180,084 and 6,264,914 and include e.g. methods used to link haptens to carrier proteins as routinely used in applied immunology (see Harlow and Lane, 1988, “Antibodies: A laboratory manual”, Cold Spring Harbor Laboratory Press, Cold Spring Harbor, NY). Given the large variety of methods for conjugation the skilled person can find a conjugation method that does not or only minimally affect the functionality of the linked entities. Suitable methods for conjugation of a ligand with an agent or carrier include e.g. carbodiimide conjugation (Bauminger and Wilchek, 1980, Meth. Enzymol. 70: 151-159). Alternatively, an agent or carrier can be coupled to a ligand as described by Nagy et al., Proc. Natl. Acad. Sci. USA 95:1794-1799 (1998). Other methods for conjugating that may suitable be used are e.g. sodium periodate oxidation followed by reductive alkylation of appropriate reactants and glutaraldehyde crosslinking. Use of active esters or Michael additions is highly preferred.

### Ligand

The ligand for the MCT is preferably a monocarboxylate. In preferred embodiments, the ligand comprises a free carboxylic acid moiety, preferably as comprised in a short chain fatty acid, an amino acid, or a keto acid. A carboxylic acid is a —COOH moiety, which depending on its environment can be its conjugated base —COO⁻, as known to a skilled person. Preferred ligands comprise a single carboxylic acid moiety. The carboxylic acid moiety is free, so it is not used for conjugation, such as via formation of an ester or amide as the donor acid. The ligand is conjugated to the nanoparticle via another site than its carboxylic acid. Because the ligand is conjugated, it can be seen as a radical of an actual ligand.

A short chain fatty acid is a fatty acids with fewer than six carbon atoms. Suitable short chain fatty acids are methanoic acid, ethanoic acid, propanoic acid, butanoic acid, 2-methylpropanoic acid, pentanoic acid, 3-methylbutanoic acid, and 2-methylbutyric acid. Examples of suitable radicals of short chain fatty acids are —X—COOH, —X—CH₂COOH, —X—CH₂CH₂COOH, —X—CH₂(CH₂)₂COOH, —X—C(CH₃)₂COOH, —X—CH₂CH(CH₃)COOH, —X—CH₂(CH₂)₃COOH, —X—C(CH₃)₂CH₂COOH, —X—CH₂CH(CH₃)CH₂COOH, —X—CH₂CH₂CH(CH₃)COOH; more preferred are —X—CH₂COOH, —X—CH₂CH₂COOH, —X—CH₂(CH₂)₂COOH, —X—C(CH₃)₂COOH, —X—CH₂CH(CH₃)COOH, —X—CH₂(CH₂)₃COOH, —X—C(CH₃)₂CH₂COOH, —X—CH₂CH(CH₃)CH₂COOH, —X—CH₂CH₂CH(CH₃)COOH; even more preferred are more preferred are —X—CH₂CH₂COOH, —X—CH₂(CH₂)₂COOH, —X—C(CH₃)₂COOH, —X—CH₂CH(CH₃)COOH, —X—CH₂(CH₂)₃COOH, —X—C(CH₃)₂CH₂COOH, —X—CH₂CH(CH₃)CH₂COOH, —X—CH₂CH₂CH(CH₃)COOH, wherein X is either absent or is a heteroatom, preferably O, N(H), Se, or S, most preferably S. In some embodiments X is S. In some embodiments X is not absent. A preferred fatty acid in this context is —X—CH₂(CH₂)₂COOH.

Amino acids are widely known. Preferred amino acids are short chain fatty acids that comprise an —NH₂ at the carbon atom directly adjacent to its carbonyl moiety. Examples of suitable radicals of ligands are —X—CH(NH₂)COOH, —X—CH₂CH(NH₂)COOH, —X—(CH₂)₂CH(NH₂)COOH, —X—CH₂C(NH₂)(CH₃)COOH, —X—C(CH₃)₂CH(NH₂)COOH, —X—CH₂CH(CH₃)CH(NH₂)COOH, and —X—CH₂CH₂C(NH₂)(CH₃)COOH, wherein X is either absent or is a heteroatom, preferably O, N(H), Se, or S, most preferably S. In some embodiments X is S. In some embodiments X is not absent. Amino acids are preferably L-amino acids. When X is S, —X—CH₂CH(NH₂)COOH can be seen as cysteine, which is a highly preferred amino acid in this context.

A keto acid is a carboxylic acid that also comprises an oxo moiety. Preferred keto acids are keto acids where the oxo moiety is on the carbon atom directly adjacent to the carboxylic acid moiety. Examples of suitable radicals of keto acids are —X—C(═O)COOH, —X—CH₂C(═O)COOH, —X—CH₂CH₂C(═O)COOH, —X—CH₂(CH₂)₂C(═O)COOH, —X—C(CH₃)₂C(═O)COOH, and —X—CH₂CH(CH₃)C(═O)COOH; more preferably —X—CH₂C(═O)COOH, —X—CH₂CH₂C(═O)COOH, —X—CH₂(CH₂)₂C(═O)COOH, —X—C(CH₃)₂C(═O)COOH, and —X—CH₂CH(CH₃)C(═O)COOH, wherein X is either absent or is a heteroatom, preferably O, N(H), Se, or S, most preferably S. In some embodiments X is S. In some embodiments X is not absent. A highly preferred keto acid is —X—CH₂C(═O)COOH in this context.

Preferably, the ligand is a ligand that specifically binds to or is taken up into a retinal cell at a rate that is at least 10% enhanced as compared to control conditions selected from a) uptake in cells lacking expression of monocarboxylate transporter; b) uptake in cells pre-treated with MCT inhibitors; and c) uptake of a reference conjugate lacking a ligand for a monocarboxylate transporter; when measured at 2 hours or more after contacting the ligand with the target cell.

As used herein, the term “specific binding” means binding that is measurably different from a non-specific interaction. Specific binding can be measured, for example, by determining binding of a molecule (ligand) compared to binding of a control molecule (ligand), which generally is a molecule of similar structure that does not have (specific) binding activity, for example, a peptide of similar size that lacks a specific binding sequence. Specific binding is present if a ligand has measurably higher affinity for the receptor than the control ligand. Specificity of binding can be determined, for example, by competition with a control ligand that is known to bind to a target. The term “specific binding,” as used herein, includes both low and high affinity specific binding. Specific binding can be exhibited, e.g., by a low affinity targeting agent having a Kd of at least about 10⁻⁴ M. E.g., if a receptor has more than one binding site for a ligand, a ligand having low affinity can still be useful. Specific binding also can be exhibited by a high affinity ligands, e.g. a ligand having a Kd of at least about of 10⁻⁷ M, at least about 10⁻⁸ M, at least about 10⁻⁹ M, at least about 10⁻¹⁰ M, or can have a Kd of at least about 10⁻¹¹ M or 10⁻¹² M or greater. Both low and high affinity-targeting ligands are useful for incorporation in the conjugates of the present invention.

Specific binding or uptake are preferably enhanced by at least 20%, 30%, 40%, 50%, 60%, 70%, 80%, 90%, 100%, 150%, 200%, 250%, 300%, 350%, 400%, or more, more preferably at least 100% or more, most preferably at least 250% or more. Enhancement is preferably measured at 2, 3, 4, 5, 6, 7, 8 or more hours after contacting the ligand with the target cell.

Cells lacking expression of monocarboxylate transporter are for instance pancreatic beta cells, or cells that have been treated to reduce or eliminate expression of MCTs, for instance via RNA interference techniques. Cells pre-treated with MCT inhibitors are preferably pre-treated with AZD3965 and AR-C155858 or with other MCT inhibitors known in the art. A reference conjugate lacking a ligand for a monocarboxylate transporter is preferably a conjugate that differs only in the absence or presence of the ligand. An example is the use of untargeted liposomes (Lp-OMe) in table S3 of the examples.

In preferred embodiments, the ligand is of general formula (I):

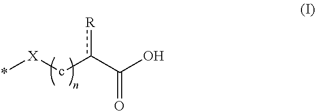

wherein
X is S, O, Se, or NH; preferably X is S or Se, more preferably S; in some embodiments X is S, O, or NH, in other embodiments X is S or O;
c is —CH₂—, —CH(CH)—, —C(═O)—, —C(═S)—, —C(═NH)—, —CH(—OH)—, —CH(NH₂)—, —CH(halogen)-, or —C(halogen)₂-; preferably c is —CH₂—, —CH(CH₃)—, —C(═O)—, or —CH(NH₂)—; most preferably c is —CH₂—;
n is 1, 2, or 3; preferably n is 1 or 2, most preferably n is 1; and
R is —H, —CH₃, ═O, ═S, ═NH, —OH, —NH₂, or a halogen; preferably R is —H, ═O, ═S, ═NH, —OH, or —NH₂; more preferably R is ═O, ═S, ═NH, —OH, or —NH₂; even more preferably R is ═O, ═S, ═NH, or —NH₂; still more preferably R is =O, ═S, or —NH₂; most preferably it is =O or —NH₂; and
is the site of conjugation to the pharmaceutically acceptable nanoparticle.

The [symbol] bond in general formula (I) is a double or a single bond, depending on the nature of R and the related requirements for valency. Herein, halogen is preferably F, Cl, Br, or I, more preferably F or Cl, most preferably F. Preferably, when more than one instance of c is present, at least one of them is —CH₂—. In some embodiments R is =O, ═S, or =NH. In some embodiments R is —OH or —NH₂. In some embodiments R is ═O. In some embodiments R is —NH₂.

In preferred embodiments X is S or O, preferably S;
c is —CH₂—, —CH(CH₃)—, —C(═O)—, or —CH(NH₂)—; most preferably —CH₂—;
n is 1 or 2, most preferably 1; and
R is =O, ═S, or —NH₂; most preferably =O or —NH₂; and;
is the site of conjugation to the pharmaceutically acceptable nanoparticle.

The means of conjugation is not critical and conjugation can be implemented however a skilled person sees fit. For instance, the nanoparticle or a constituent substance thereof can comprise a leaving group or a Michael acceptor or a cycle that can be opened by a nucleophile of the ligand, or it can comprise a carboxylic acid or amine or thiol or hydroxyl moiety that can be reacted via the formation of an amide or an ester or a dithiol bridge. For instance, when X is S in ligands as described above, the free ligand can have an —SH moiety, which is highly versatile for conjugation. Similarly, when X is O, the free ligand can have a corresponding —OH, and when X is NH, the free ligand can have —NH₂. A skilled person can select a suitable means of conjugation. Preferred examples are nanoparticles comprising Michael acceptors, active esters, or alfa-halogenic acetic acid moieties on their surface. Preferred Michael acceptors are maleimides, vinylketones, acrylic moieties, or methacrylic moieties. Preferred active esters are esters of nitrophenol, pentafluorophenol, or N-hydroxysuccinimide (NHS). Preferred alfa-halogenic acetic acid moieties are esters or amides of iodoacetic acid or of bromoacetic acid. In preferred embodiments the ligand is conjugated to the water soluble polymer as described later herein.

### Monocarboxylate Transporter (MCT)

MCTs are a family of proton-linked plasma membrane transporters that carry molecules having one carboxylate group (monocarboxylates) across biological membranes. They belong to the group of solute carrier (SLC) membrane transport proteins. Suitable molecules for transport by MCTs are lactate, pyruvate, and ketones. MCTs are widely expressed. Highly malignant tumors rely heavily on anaerobic glycolysis (metabolism of glucose to lactic acid even under presence of oxygen; Warburg effect) and thus efflux lactic acid via MCTs to the tumor micro-environment to maintain a robust glycolytic flux and to prevent detrimental accumulation of lactic acid inside the tumor. At least 14 MCTs are known, corresponding to 14 solute carrier 16A transporters, namely SLC16A1, SLC16A2, SLC16A3, SLC16A4, SLC16A5, SLC16A6, SLC16A7, SLC16A8, SLC16A9, SLC16A10, SLC16A11, SLC16A12, SLC16A13, and SLC16A14.

In some embodiments, the MCT is at least one of MCT1, MCT2, MCT3, MCT4, MCT5, MCT6, MCT7, MCT8, MCT9, MCT10, MCT11, MCT12, MCT13, and MCT14. In preferred embodiments, the monocarboxylate transporter is at least monocarboxylate transporter 1 (MCT1), monocarboxylate transporter 2 (MCT2), monocarboxylate transporter 3 (MCT3), or monocarboxylate transporter 4 (MCT4). In more preferred embodiments, the monocarboxylate transporter is at least monocarboxylate transporter 1 (MCT1), monocarboxylate transporter 2 (MCT2), or monocarboxylate transporter 4 (MCT4), more preferably each of MCT1, MCT2, and MCT4. In particular embodiments, the monocarboxylate transporter is at least monocarboxylate transporter 3 (MCT3). In particular embodiments, the monocarboxylate transporter is at least monocarboxylate transporter 4 (MCT4). In highly preferred embodiments, the monocarboxylate transporter is at least monocarboxylate transporter 1 (MCT1) or monocarboxylate transporter 2 (MCT2), more preferably both MCT1 and MCT2.

### Pharmaceutically Acceptable Nanoparticle

The invention lies in the surprising finding that nanoparticles such as nanocarriers can effectively target themselves and their cargo to photoreceptors. With this insight a skilled person can select a suitable pharmaceutically acceptable nanoparticle. In preferred embodiments the pharmaceutically acceptable nanoparticle is a liposome, a solid lipid nanoparticle, a micelle, a carrier protein, a metal nanoparticle, a polyplex system, a lipoplex system, or a polymeric nanoparticle. More preferably it is a liposome, a solid lipid nanoparticle, a micelle, a carrier protein, or a polymeric nanoparticle. Still more preferably it is a liposome, a solid lipid nanoparticle, a micelle, or a carrier protein, and even more preferably it is a liposome, a solid lipid nanoparticle, or a micelle. In particular embodiments, the nanoparticles is a liposome, a micelle, a carrier protein, a polyplex system, a lipoplex system, or a polymeric nanoparticle.

Good results were achieved with lipid nanoparticles. Accordingly, a preferred pharmaceutically acceptable nanoparticle is a lipid nanoparticle. Lipid nanoparticles can be liposomes, solid lipid nanoparticles, and lipid micelles. Lipid nanoparticles can also be lipid polymer hybrid nanoparticles. Preferred polymers are biodegradable polymers such as polylactic acid. In some embodiments the lipid nanoparticles are liposomes or lipid micelles. In some embodiments the lipid nanoparticles are lipid micelles or solid lipid nanoparticles. In some embodiments the lipid nanoparticles are solid lipid nanoparticles or liposomes. Liposomes are most preferred.

A nanoparticle is herein understood to be a small object that behaves as a single integer unit with respect to its transport and properties. Preferably, when a nanoparticle of the invention is a solid lipid nanoparticle, it does not comprise any stabilizing surfactants. The size of a lipid nanoparticle is preferably between 1 and 300 nm.

The pharmaceutically acceptable nanoparticle preferably comprises a lipid; preferably at least two lipids. In preferred embodiments the pharmaceutically acceptable nanoparticle comprises one or more phospholipids. Phospholipids preferably contain a diglyceride, a phosphate group and a simple organic molecule such as choline. In particular, “phospholipids” include phosphatidylcholine (PC), phosphatidylethanolamine (PE), phosphatidic acid (PA), phosphatidylinositol (PI), phosphatidylserine (PS), sphingomyelin, plasmalogens, and phosphatidylcholine lipid derivatives where the two hydrocarbon chains are typically between about 14-22 carbon atoms in length, and have varying degrees of unsaturation.

The phospholipid may comprise a net negative electrical charge or a net positive electrical charge. However in a preferred embodiment of the invention, one or more phospholipids are neutral phospholipids. More preferably all phospholipids are neutral phospholipids. A neutral phospholipid is herein understood as a phospholipid that has no net electrical charge.

In a preferred embodiment, the lipid comprises one or more neutral phospholipids selected from the group consisting of 1,2-dilauroyl-sn-glycero-3-phosphate (DLPA), 1,2-dilauroyl-sn-glycero-3-phosphoethanolamine (DLPE), 1,2-dimyristoyl-sn-glycero-3-phosphate (DMPA), 1,2-dimyristoyl-sn-glycero-3-phosphocholine (DMPC), 1,2-dimyristoyl-sn-glycero-3-phosphoethanolamine (DMPE), 1,2-dimyristoyl-sn-glycero-3-phosphoglycerol (DMPG), 1,2-dimyristoyl-sn-glycero-3-phosphoserine (DMPS), 1,2-dipalmitoyl-sn-glycero-3-phosphate (DPPA), 1,2-dipalmitoyl-sn-glycero-3-phosphocholine (DPPC), 1,2-dipalmitoyl-sn-glycero-3-phosphoethanolamine (DPPE), 1,2-dipalmitoyl-sn-glycero-3-phosphoglycerol (DPPG), 1,2-dipalmitoyl-sn-glycero-3-phosphoserine (DPPS), 1,2-distearoyl-sn-glycero-3-phosphate (DSPA), 1,2-distearoyl-sn-glycero-3-phosphocholine (DSPC), 1,2-distearoyl-sn-glycero-3-phosphoethanolamine (DSPE), 1,2-distearoyl-sn-glycero-3-phosphoglycerol (DSPG), 1,2-distearoyl-sn-glycero-3-phosphoserine (DSPS) and hydrogenated soy phosphatidylcholine (HSPC). In addition, the one or more neutral phospholipids for use in the invention may be soy phosphatidylcholine (SPC) or egg yolk phosphatidylcholine (EYPC) or 1-palmitoyl-2-oleoyl-sn-glycero-3-phosphocholine (POPC). However, SPC and EYPC may be less preferred as lipids for the (solid lipid) nanoparticle, as these lipids may limit the stability of the lipid nanoparticle, potentially because of the melting point of SPC and EYPC is below room temperature. In some embodiments the one or more phospholipid is selected from DSPC and DSPE and POPC. In some embodiments the nanoparticles comprise DSPE and POPC. In some embodiments the nanoparticles comprise DSPE and DSPC. Nanoparticles such as liposomes containing DSPC can exhibit slower drug release and are expected to be better suited for in vivo applications. DSPE is advantageously comprised in nanoparticles as part of a conjugate of DSPE and a water soluble polymer such as PEG.

A lipid nanoparticle may comprise a single or a variety of lipids. In particular, the nanoparticle according to the invention may comprise at least 1, 2, 3, 4, or 5 different lipids. Alternatively, the nanoparticle may comprise at most 1, 2, 3, 4, or 5 different lipids. It is preferred that lipid nanoparticles comprise two or more lipids, such as three different lipids, particularly for liposomes. In some embodiments only a single lipid is comprised, which is particularly attractive for micelles. Lipids are preferably biocompatible lipids.

In some embodiments the pharmaceutically acceptable nanoparticle comprises one or more non-cationic lipids. A preferred non-cationic lipid is a sterol, more preferably cholesterol or a derivative thereof, most preferably cholesterol. The total amount of lipid preferably comprises at least about 1, 5, 10, 20, 25, 30, 35, 40 or 45% cholesterol (derivative) (w/w). Preferably, the lipid comprises at least 25 or 30% cholesterol (derivative), such as about 30-35%. Alternatively, the molar ratio between a phospholipid and a non-cationic lipid is in the range of 0.2:1 to 5:1, preferably 1:1 to 4:1, even more preferably 1.5:1 to 2.5:1, with a ratio of about 2:1 being particularly preferred in view of good results. Alternatively, in some embodiments the lipid comprises less than 0.1, 0.5, 1, 5, 10, 20, 30, 35, 40 or 45% cholesterol (derivative) (w/w).

A (stabilising) water soluble polymer is herein understood as a polymer that contributes to the stability to the nanoparticle e.g. in vivo by increasing the tissue penetration/diffusion depth, the circulation time and/or decreasing bloodstream clearance (so-called “stealth” properties) as compared to the same nanoparticle without the water soluble polymer. The water soluble polymer for use in the invention may alternatively or in addition have a lubricating effect. The water soluble polymer may be a stabilising and/or moisturizing water soluble polymer.

A liposome is a lipid nanoparticle comprising a lipid bilayer and an aqueous interior or aqueous lumen. Liposomes are attractive for delivering hydrophilic pharmaceutically active agents because these can be encapsulated in the lumen. Liposomes can have multiple bilayers, forming multilamellar vesicles. The lumen can comprise further vesicles, forming multivesicular vesicles. A solid lipid nanoparticle is a lipid nanoparticle that lacks an aqueous interior. It may be multi-layered, or it may be unstructured. A solid lipid nanoparticle is attractive for delivering hydrophobic pharmaceutically active agents. A micelle is preferably a lipid micelle and is attractive for delivering hydrophobic pharmaceutically active agents; a micelle is conveniently formed out of a single surfactant such as a single phospholipid, or a single conjugate of a lipid and a water soluble polymer; a micelle can also be formed out of a phospholipid and a polymer-lipid conjugate as described herein; a micelle can also be formed out of a non-cationic lipid and a polymer-lipid conjugate as described herein. A carrier protein can be any protein suitable for being conjugated to the ligand, preferably while comprising (for instance via conjugation, encapsulation or hydrophobic interactions) a pharmaceutically active agent. A metal nanoparticle can for example be a contrast agent or an anticancer agent or a diagnostic agent such as described by Liang et al. (doi: 10.2147/IJN.S75174). A polyplex can be a complex of a nucleic acid with a polymer such as polylysine or polyethylene imine. A lipoplex can be a complex of a nucleic acid with lipids. A polymeric nanoparticle can be a layer-by-layer particle, a polymersome, or a solid polymer nanoparticle, for instance as prepared by suspension polymerisation; it is preferably a biodegradable polymeric nanoparticle, based on for instance polylactic acid. Polymeric nanoparticles can be hybrid nanoparticles that also comprise lipids.

In preferred embodiments the pharmaceutically acceptable nanoparticle comprises a water soluble polymer at its surface. Thus, the nanoparticle preferably has a surface comprising a water soluble polymer. Preferably, the surface of the nanoparticle is at least partly covered by the water soluble polymer. More preferably, the water soluble polymer covers the surface of the nanoparticle for at least about 10, 20, 30, 40, 50, 60, 70, 80, 90, 99 or 100%. In a further embodiment, the nanoparticle has a surface consisting of a water soluble polymer.

In a further embodiment, the lipid nanoparticle for use according to the invention comprises a water soluble polymer, wherein the water soluble polymer is at least one of:
i) a polyalkylether, preferably the polyalkylether is linear polyethylene glycol (PEG), star PEG or multi-arm branched PEG;
ii) a homopolymer that is a PEG substitute or a PEG alternative, preferably the homopolymer is selected from the group consisting of polymethylethyleneglycol (PMEG), polyhydroxypropyleneglycol (PHPG), polypropyleneglycol (PPG), polyvinylpyrrolidone (PVP), polyglycerol (PG), polymethylpropyleneglycol (PMPG), polyhydroxypropyleneoxide (PHPO), poly-oxazoline (POZ), and hydroxyethyl starch (HES);
iii) a heteropolymer of small alkoxy monomers, preferably the heteropolymer comprises polyethylene/polypropyleneglycol (PEG/PPG).

PEG is most preferred, and is also known as polyethylene oxide (PEO) or polyoxyethylene (POE), depending on its molecular weight and these names may be used interchangeable herein. The water soluble polymer may confer stealth-like and/or moisturizing and/or stabilizing properties to the nanoparticle.

The nanoparticle may comprise a single or a variety of water soluble polymers. In particular, the nanoparticle may comprise at least 1, 2, 3, 4, 5, 6, 7, 8, 9 or 10 different water soluble polymers. Alternatively, the nanoparticle comprise at most 1, 2, 3, 4, 5, 6, 7, 8, 9 or 10 different water soluble polymers. Furthermore, instead of or in addition to the water soluble polymer as defined herein, the water-soluble polymer may be a derivative of the above-defined polyalkylether, homopolymer and/or heteropolymer. In particular, the water-soluble polymer may be derivatised to comprise a functional group such as for example a carboxylic acid, a maleimide, or an amide for e.g. covalently linking the ligand.

In a preferred embodiment, the water soluble polymer has a molecular weight of at least about 120 Daltons and up to 20,000 Da. Preferably, the water soluble polymer has a molecular weight between 300 and 10,000 Da, more preferably between 500 and 5,000 Da, and most preferably between 750 and 3,000 Da or between 1,000 and 3,400 Da, or between 1,000 and 2,000 Da, e.g. around 2000 Da.

In another preferred embodiment, the water soluble polymer has a molecular weight that is less than 20,000, 15,000, 10,000, 5,000, 4,500, 4,000, 3,500, 3,400, 3,300 or 3,200 Da. In a further preferred embodiment, the water soluble polymer has a polymerization number of at least about 4, 5, 6, 7, 8, 9, 10, 25, 50, 75, 100, 125, 150, 175, 200, 209, 210, 211, 250, 300, 400 or 500. In particular, preferably the water soluble polymer has a polymerization number of at least 6 or about 6-210. In a further embodiment, the water soluble polymer is conjugated to one of the lipids, or to the lipid. Preferably, the water soluble polymer is covalently linked. In a particularly preferred embodiment, the conjugate of the lipid and the water soluble polymer is a conjugate of a phospholipid as defined herein, to a polymer as defined above.

In a preferred embodiment, a lipid such as a phospholipid may be conjugated or linked to a water soluble polymer. Such polymer-phospholipid conjugates may have a stabilising effect on the nanoparticle, and a moisturizing and/or lubricating effect. Highly suitable for being comprised in such a polymer-lipid conjugate is 1,2-distearoyl-sn-glycero-3-phosphoethanolamine (DSPE), most preferably N-linked. More preferably, the conjugate is a 1,2-distearoyl-sn-glycero-3-phosphoethanolamine-N-polyethylene glycol (DSPE-PEG), and most preferably wherein the conjugate is 1,2-distearoyl-sn-glycero-3-phosphoethanolamine-N-[methoxy(polyethylene glycol)-2000] (DSPE-mPEG2000) or d-alpha tocopheryl-N-[methoxy(polyethylene glycol)-1000] (TPEG1000). In an alternative embodiment of the invention, the conjugate is hydrogenated soy phosphatidylcholine-polyethylene glycol (HSPC-PEG), and most preferably wherein the HSPC-PEG is hydrogenated soy phosphatidylcholine-[methoxy(polyethylene glycol)-2000](HSPC-mPEG2000).

Such a polymer-lipid conjugate is preferably present in the nanoparticle in an amount ranging from 0.5 to 15% by total weight of lipid. Herein the weight of the polymer-lipid conjugate is considered as contributing in its entirety to total lipid weight. More preferably it is present in an amount ranging from 1 to 10% by total weight of lipid, still more preferably 2 to 8%, more preferably 3 to 7%, most preferably 4 to 6% such as about 5%.

In lipid nanoparticles, particularly in liposomes, preferably a phospholipid, a non-cationic lipid, and a polymer-lipid conjugate are present, in weight ratios in the range of 1-36:1-18:0.1-3, preferably in the range of 6-24:3-12:0.5-2, more preferably 8-16:4-8:0.7-1.3, such as about 12:6:1. Most preferably it is about 63.3:31.7:5.

In an embodiment of the invention, the size of the lipid nanoparticle is between 5-1000 nm, preferably the size of the nanoparticle is between 15 and 500 nm and more preferably the size of the nanoparticle is between 20 and 250 nm, more preferably between 30 and 200, still more preferably between 40 and 100, or between 50 and 90 nm. PEGylated liposomes less than 100 nm in diameter are likely to reach photoreceptors. The size of the nanoparticle may be determined by any method known in the art. Preferably the size of the nanoparticle is determined by dynamic light scattering zeta-sizer.

Preferably, for solid lipid nanoparticles or micelles, the size of the nanoparticle is at least about 3, 5, 10, 15, 20, 25, 30, 35, 40, 45 or 50 nm and not more than about 350, 300, 250, 200, 175, 150, 125, 100, 75 or 50 nm. More preferably the size of the nanoparticle is about 5, 10, 15, 20, 25, 30, 35, 40, 45, 50, 55, 60, 65, 70, 75, 80, 85, 90, 95 or 100 nm. Most preferably, the size of the nanoparticle is about between 5 and 300 nm, 10 and 150 nm, 15 and 100 nm, 20 and 100 nm, 15 and 80 nm, 20 and 80 nm, 15 and 60 nm or 20 and 60 nm.

In a further preferred embodiment, the invention relates to a composition comprising conjugates of the invention, further comprising a pharmaceutically acceptable adjuvant such as water. Preferably, in such a composition, the nanoparticles have an average size of at least about 3, 5, 10, 15, 20, 25, 30, 35, 40, 45 or 50 nm and not more than about 350, 300, 250, 200, 175, 150, 125, 100, 75 or 50 nm. More preferably the average size of the nanoparticles is about 5, 10, 15, 20, 25, 30, 35, 40, 45, 50, 55, 60, 65, 70, 75, 80, 85, 90, 95 or 100 nm. The nanoparticles in the composition may deviate at least 0.01, 0.05, 0.10, 0.15, 0.20, 0.25, 0.30, 0.35, 0.40, 0.45, 0.50, 0.60, 0.70, 0.80, 0.90, 1.0, 2.0 or 5.0 nm from the average size. Most preferably, the size of the nanoparticles in the composition is about between 5 and 300 nm, 10 and 150 nm, 15 and 100 nm, 20 and 100 nm, 15 and 80 nm, 20 and 80 nm, 15 and 60 nm, 20 and 60 nm, 15 and 50 nm, 20 and 50 nm, 15 and 40 nm, 20 and 40 nm, 15 and 30 nm or 20 and 30 nm.

A nanoparticle for the invention can be obtained using any method known in the art. A mixture comprising the nanoparticle may subsequently be sterilized using any conventional method. For example, the mixture comprising conjugates of the invention may be sterilized by passing the mixture though a (sterile) filtration filter. Preferably, the filtration filter comprises a pore size of about 0.15, 0.2, 0.25, 0.3, 0.4, 0.5, 0.5 or 1.0 microns.

A conjugate or nanoparticle as disclosed herein may be prepared using any conventional method known in the art. As a non-limiting example, the nanoparticle may be prepared by dissolving a lipid, a water-soluble polymer and a diagnostic, lubricating or therapeutic agent in any suitable solvent. Preferred solvents are miscible in water, and are pharmaceutically acceptable. Particularly preferred solvents are ethanol, methanol and isopropanol, more preferably ethanol and methanol. The most preferred solvent is ethanol. The solution may be heated. Preferably, the solution is heated to about 40, 55, 60, 65 or 70 degrees Celsius. Subsequently the lipid solution may be added to a suitable aqueous solution, preferably having about the same temperature as the lipid solution. The lipid solution is preferably added slowly, e.g. step-wise. Alternatively, an aqueous solution may be added to the lipid solution. Preferably, the aqueous solution is added slowly, e.g. step-wise. A suitable aqueous solution includes water, saline, phosphate buffered saline, or any other aqueous solution commonly known in the art. A preferred aqueous solution is water. The percentage solvent/aqueous solution is preferably about 5%, 10%, 15%, 20%, 25%, 30%, 35%, 40%, 45%, 50%, 55% or 60% (w/w). After the solution is cooled down, any residual solvent may optionally be removed from the particle solution. Removal of the residual solvent can be done using any conventional method known in the art including, but not limited to, dialysis, diafiltration or liquid chromatography.

Alternately, liposomes can be prepared using a thin-film rehydration method. Here, a chloroform solution of the lipids such as POPC, cholesterol, and a phospholipid-PEG conjugate can be mixed in a molar ratio of for example 63.3:31.7:5 to produce untargeted liposomes with PEG chains on their surface. For pharmaceutically active agent encapsulation, the lipid DSPC can be used instead of POPC. All lipid solutions can be dried, such as with a rotation evaporator under reduced vacuum (for instance 300 mbar). After about 1 h, the dried lipids can be rehydrated in either a rehydration buffer. Agents can be added in for instance a 1:3 drug-to-lipid molar ratio. After lipid dissolution in the rehydration medium, about 5 freeze-thaw cycles were performed in liquid nitrogen and 37° C. water bath. The liposome solutions can be extruded for instance about 11 times through a PC membrane with about 100 nm pores. For conjugation with MCT ligands, suitable nucleophilic analogues such as thiols can be prepared in 10 mM of a reducing agent such as tris(2-carboxyethyl)phosphine in a buffer such as 25 mM HEPES (pH 7.4) and can be added to Michael acceptor containing nanoparticles such as PEG-maleimide-containing liposomes, using ligand at twice the maleimide concentration, followed by incubation at room temperature for 2 h. To remove non-encapsulated compounds and unbound ligands, the formulations can be dialyzed for instance against isotonic saline at 4° C. For removal of agents, a 2 h dialysis period can be performed. The nanoparticles such as liposomes can be sterile filtered and stored at for instance 2-8° C.

### Cargo

In some embodiments the invention provides the conjugate according to the invention, wherein the pharmaceutically acceptable nanoparticle further comprises a pharmaceutically active agent, preferably a neuroprotective agent, such as a photoreceptor rescuing drug, preferably a cyclic guanosine monophosphate (cGMP) analogue. Nanoparticles such as lipid nanoparticles comprising pharmaceutically active agents are widely known. Examples of suitable pharmaceutically active agents are:
a. a neuroprotective agent;
b. a central nervous system depressant agent;
c. a central nervous system stimulant agent;
d. a psychopharmacological agent;
e. a respiratory tract drug;
f. a peripheral nervous system drug;
g. a drug acting at synaptic or neuroeffector junctional sites;
h. a smooth muscle active drug or a skeletal or cardiac muscle actice drug;
i. a histaminergic agent;
j. an antihistaminergic agent;
k. a cardiovascular drug;
l. a blood or hemopoietic system drug;
m. a gastrointestinal tract drug;
n. a steroidal agent;
o. a cytostatic or antineoplastic agent;
p. an antibiotic agent;
q. an antifungal agent;
r. an antimalarial agent;
s. an antiprotozoan agent;
t. an antimicrobial agent;
u. an anti-inflammatory agent;
v. an immunosuppressive agent;
w. a cytokine;
x. an enzyme;
y. an iminosugar;
z. a ceramide analog;
aa. a brain-acting hormone or neurotransmitter;
bb. a peptide such as a neuropeptide or derivative thereof;
cc. a neurotrophic factor;
dd. an antibody or fragment thereof;
ee. an Alzheimer's Disease drug or compound;
ff. a nucleic acid such as siRNA, mRNA, or miRNA;
gg. an imaging agent;
hh. an (organophosphate) detoxifying agent;
ii. an anticancer agent.

The above pharmaceutically active agents are described in more detail in US2014/0227185 in paragraphs [0018] through [0050]. In some embodiments, the pharmaceutically active agent is a neuroprotective agent or a peptide or a nucleic acid. A pharmaceutically active agent is preferably present in a total concentration of 0.5 to 50 μM, more preferably 1 to 10 μM, even more preferably 2 to 5 μM. Examples of anticancer agents are DNA replication inhibitors such as cell-cycle nonspecific antineoplastic agents (for example cisplatin or oxaliplatin), topoisomerase inhibitors such as anthracyclines (for instance doxorubicin), mitotic inhibitors (such as paclitaxel) or combinations thereof.

More preferably, the pharmaceutically active agent is a neuroprotective agent, such as palmitoylethanolamide (PEA), sunitinib, mycophenolic acid (MPA), or a photoreceptor rescuing drug, wherein a particularly preferred photoreceptor rescuing drug is a cyclic guanosine monophosphate (cGMP) analogue. Also known as cGMP-derived PKG inhibitors, cGMP analogues (such as e.g. Rp-8-Br-cGMPS) are known to offer protection of rd1 and rd2 photoreceptors both in vitro and in in vivo mouse retinitis pigmentosa models (Paquet-Durand et al., 2009; Vighi, E., et al., PNAS, 2018, 115(13):E2997-E3006). cGMP analogues as such are known in the art. WO2012130829 describes boranophosphate analogues of cyclic nucleotides. WO2018/010965 describes multimeric complexes of cGMP analogues. Butt et al. (FEBS letters, 1990, 263(1): 48, DOI: 10.1016/0014-5793(90)80702-K) describe inhibition of cGMP-dependent protein kinase by (Rp)-guanosine 3′,5′-monophosphorothioates.

Most preferably the pharmaceutically active agent is a cGMP analogue. Preferably the cGMP analogue is of general formula (cGMP) or a salt thereof:

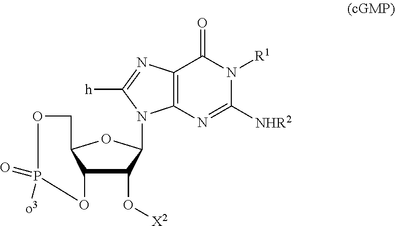

wherein X² is p′ of H, wherein p′ is a hydroxyl protective group, preferably methoxymethyl (MOM), tetrahydropyranyl (THP), t-butyl (tBu), allyl (all), benzyl (Bn), (tri)alkylsilyl (such as t-butyldimethylsilyl (TBDMS), triisopropylsilyl (TIPS), or t-butyldiphenylsilyl (TBDPS)), acyl (such as acetyl (Ac), pivaloyl (Pv), or benzoyl (Bz));
h is H, halogen, or Q;
R¹ and R² are each independently chosen from H, —(CH₂)ₙ—H, —(CH₂)ₙ—C₃₋₉heterocyclyl, —(CH₂)ₙ-ar, and ar, wherein each instance of n is independently chosen from 0, 1, 2, 3, or 4, or R¹ and R² together form —CH═C(ar)- or —(CH₂)₁₋₄C(═O)—;
ar is in each instance independently a 5- or 6-membered aromatic or heteroaromatic ring, preferably phenyl or 2-furanyl, wherein each instance of ar is individually optionally substituted with halogen, —OH, —SH, —NH₂, —NO₂, —OCH₃, —CH₃, —CH₂CH₃, —CH(CH₃)₂, or —CF₃, and is optionally fused with a second instance of ar, preferably forming a naphthyl moiety;
Q is —(CH₂)ₙ—S—(CH₂)ₙ—H, —S—(CH₂)ₙ—OH, —S—(CH₂)ₙ—NH₂, —(CH₂)ₙ—O—(CH₂)ₙ—H, —O—(CH₂)ₙ—OH, —O—(CH₂)ₙ—NH₂, —O—C(CH₃)₃, —O—CH(CH₃)₂, —(CH₂)ₙ—N(—[CH₂]ₙH)₂, —NH—(CH₂)ₙNH₂, —NH—(CH₂)ₙ—OH, —(CH₂)ₙ-Nc¹c² wherein c¹ and c² together with the N to which they are attached form a 3 to 8 membered heterocycle or wherein c¹ is H and c² is a 3 to 8 membered heterocycle, —(CH₂)ₙ—H, —N₃, —CF₃, —(CH₂)ₙ-ar, —O—(CH₂)ₙ-(ar), —NH—(CH₂)ₙ-(ar), —S—(CH₂)ₙ-(ar), —(CH₂)ₙ-amido-ar, —O—(CH₂)ₙ-amido-(ar), —NH—(CH₂)ₙ-amido-(ar), —S—(CH₂)ₙ-amido-(ar), or a linker moiety, wherein any —H may be optionally replaced by a halogen, wherein each instance of n is independently chosen from 0, 1, 2, 3, 4, 5, 6, 7, or 8; and
o³ is H, —SH, or —S—C₁-12hydrocarbon, borano, methylborano, dimethylborano, or cyanoborano, preferably o³ is —SH, borano, methylborano, dimethylborano, cyanoborano. Most preferably —SH.

Preferably the cGMP analogue of general formula (cGMP) is of general formula (cGMP-Rp):

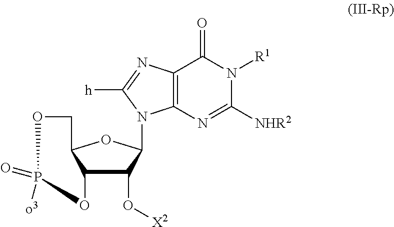

o³ is SH, borano, methylborano, dimethylborano, cyanoborano. Said boron analogues, however, are referred to as Sp-analogues due to lower priority of boron compared to oxygen within Cahn-Ingold-Prelog nomenclature rules.

Further preferred examples of cGMP analogues are:
1. 8-Bromoguanosine-3′, 5′-cyclic monophosphate (8-Br-cGMP) or its phosphorothioate (8-Br-cGMPS),
2. 8-(2, 4-dihydroxyphenylthio)guanosine-3′, 5′-cyclic monophosphate (8-o,pDHPT-cGMP) or its phosphorothioate 8-o,pDHPT-cGMPS,
3. 8-(2-aminophenylthio)guanosine-3′, 5′-cyclic monophosphate (8-APT-cGMP) or its phosphorothioate 8-APT-cGMPS,
4. 8-(4-hydroxyphenylthio)guanosine-3′, 5′-cyclic monophosphate (8-pHPT-cGMP) or its phosphorothioate 8-pHPT-cGMPS,
5. 8-(4-aminophenylthio)guanosine-3′, 5′-cyclic monophosphate (8-pAPT-cGMP) or its phosphorothioate 8-pAPT-cGMPS,
6. 8-(4-chlorophenylthio)-3-phenyl-1,N²-ethenoguanosine-3′,5′-cyclic monophosphate (8-pCPT-PET-cGMP) or its phosphorothioate 8-pCPT-PET-cGMPS,
7. 8-(4-chlorophenylthio)guanosine-3′, 5′-cyclic monophosphate (8-pCPT-cGMP) or its phosphorothioate 8-pCPT-cGMPS,
8. 8-(2, 4-dichlorophenylthio)guanosine-3′, 5′-cyclic monophosphate (8-o,pDCIPT-cGMP) or its phosphorothioate 8-o,pDCIPT-cGMPS,
9. 8-(4-methoxyphenylthio)guanosine-3′, 5′-cyclic monophosphate (8-pMeOPT-cGMP) or its phosphorothioate 8-pMeOPT-cGMPS,
10. 8-bromo-β-phenyl-1, N²-ethenoguanosine-3′, 5′-cyclic monophosphate (8-Br-PET-cGMP) or its phosphorothioate 8-Br-PET-cGMPS,
11. 8-bromo-(2-naphthyl-1, N²-etheno)guanosine-3′, 5′-cyclic monophosphate (8-Br-(2-N)ET-cGMP) or its phosphorothioate 8-Br-(2-N)ET-cGMPS,
12. 8-(4-hydroxyphenylthio)-3-phenyl-1,N²-ethenoguanosine-3′,5′-cyclic monophosphate (8-pHPT-PET-cGMP) or its phosphorothioate 8-pHPT-PET-cGMPS,
13. 8-(4-chlorophenylthio)-β-phenyl-1,N²-ethenoguanosine-3′,5′-cyclic monophosphate (8-pCPT-PET-cGMP) or its phosphorothioate 8-pCPT-PET-cGMPS,
14. 2-naphthyl-1, N²-ethenoguanosine-3′, 5′-cyclic monophosphate ((2-N)ET-cGMP) or its phosphorothioate (2-N)ET-cGMPS,
15. ß3-phenyl-1, N²-ethenoguanosine-3′, 5′-cyclic monophosphate (PET-cGMP) or its phosphorothioate PET-cGMPS,
16. 4-methoxy-ß-phenyl-1,N²-ethenoguanosine-3′, 5′-monophosphate (pMeO-PET-cGMP) or its phosphorothioate pMeO-PET-cGMPS,
17. β-1, N²-acetyl-8-bromoguanosine-3′, 5′-cyclic monophosphorothioate (β-1,N²—Ac-8-Br-cGMPS) and its phosphate (β-1,N²—Ac-8-Br-cGMP),
18. 8-Bromo-β-1, N²-butyrylguanosine-3′, 5′-cyclic monophosphorothioate (8-Br-δ-1,N²-But-cGMPS) and its phosphate (8-Br-δ-1,N²-But-cGMP),
19. 8-bromo-(4-methyl-β-phenyl-1, N²-etheno)guanosine-3′, 5′-cyclic monophosphorothioate (8-Br-pMe-PET-cGMPS) and its phosphate (8-Br-pMe-PET-cGMP),
20. 8-Bromo-(3-thiophen-yl-1, N²-etheno)guanosine-3′, 5′-cyclic monophosphorothioate (8-Br-(3-Tp)ET-cGMPS) and its phosphate (8-Br-(3-Tp)ET-cGMP),
21. 1-Benzyl-8-bromoguanosine-3′, 5′-cyclic monophosphorothioate (1-Bn-8-Br-cGMPS) and its phosphate (1-Bn-8-Br-cGMP),
22. 8-Thioguanosine-3′, 5′-cyclic monophosphorothioate (8-T-cGMPS) and its phosphate (8-T-cGMP),
23. 8-(4-Isopropylphenylthio)guanosine-3′, 5′-cyclic monophosphorothioate (8-plPrPT-cGMPS) and its phosphate (8-plPrPT-cGMP),
24. 8-Phenylamidomethylthioguanosine-3′, 5′-cyclic monophosphorothioate (8-PAmdMT-cGMPS) and its phosphate (8-PAmdMT-cGMP),
25. β-phenyl-1, N²-etheno-8-phenylamidomethylthioguanosine-3′, 5′-cyclic monophosphorothioate (PET-8-PAmdMT-cGMPS) & phosphate (PET-8-PAmdMT-cGMP),
26. 8-(4-Isopropylphenylthio)-β-phenyl-1, N²-ethenoguanosine-3′, 5′-cyclic monophosphorothioate (8-pIPrPT-PET-cGMPS) and its phosphate (8-pIPrPT-PET-cGMP),
27. 8-(2-Aminophenylthio)-β-phenyl-1, N²-ethenoguanosine-3′, 5′-cyclic monophosphorothioate (8-oAPT-PET-cGMPS) and its phosphate (8-oAPT-PET-cGMP),
28. β-Phenyl-1, N²-etheno-8-thioguanosine-3′, 5′-cyclic monophosphorothioate (PET-8-T-cGMPS) and its phosphate (PET-8-T-cGMP),
29. 8-Methylthio-β-phenyl-1, N²-ethenoguanosine-3′, 5′-cyclic monophosphorothioate (8-MeS-PET-cGMPS) and its phosphate (8-MeS-PET-cGMP),
30. 8-Methylthio-guanosine-3′, 5′-cyclic monophosphorothioate (8-MeS-cGMPS), preferably a sodium salt, and its phosphate (8-MeS-cGMP),
31. 8-Phenylguanosine-3′, 5′-cyclic monophosphorothioate (8-Phe-cGMPS) and its phosphate (8-Phe-cGMP),
32. 8-(2-Furyl)guanosine-3′, 5′-cyclic monophosphorothioate (8-(2-Fur)-cGMPS) and its phosphate (8-(2-Fur)-cGMP),
33. 8-(4-Chlorophenyl)guanosine-3′, 5′-cyclic monophosphorothioate (8-pCP-cGMPS) and its phosphate (8-pCP-cGMP),
34. 8-Phenyl-β-phenyl-1, N²-ethenoguanosine-3′, 5′-cyclic monophosphorothioate (8-Phe-PET-cGMPS) and its phosphate (8-Phe-PET-cGMP), and
35. 8-(4-Chlorophenyl)-β-phenyl-1, N²-ethenoguanosine-3′, 5′-cyclic monophosphorothioate (8-pCP-PET-cGMPS) and its phosphate (8-pCP-PET-cGMP),

and pharmaceutically acceptable salts thereof. A most preferred cGMP analogue is 8-Br-PET-cGMP or its phosphorothioate, preferably its phosphorothioate. Highly preferred cGMP analogues are 8-Br-PET-cGMPS and 8-pCPT-PET-cGMPS, particularly Rp-8-Br-PET-cGMPS and Rp-8-pCPT-PET-cGMPS, even more preferably their sodium salts, which are sometimes referred to as CN03 and CN04, respectively.

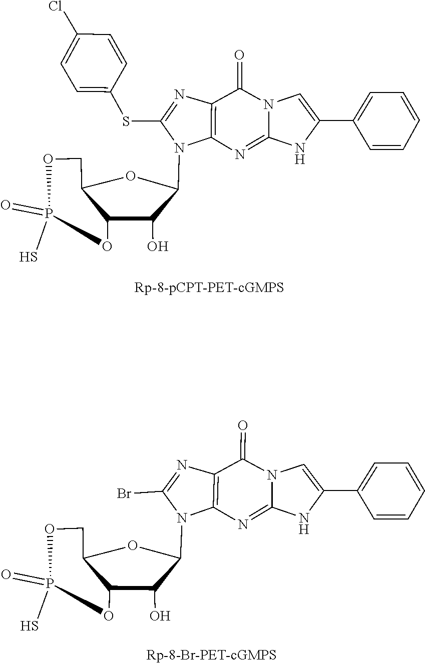

In some embodiments the pharmaceutically acceptable nanoparticle further comprises two or more pharmaceutically active agents. In some embodiments the pharmaceutically active agent is comprised in the aqueous lumen of the pharmaceutically acceptable nanoparticle. In some embodiments the pharmaceutically active agent is comprised in a solid phase of the pharmaceutically acceptable nanoparticle, such as in a lipid phase or in a lipid bilayer. A skilled person can select a suitable dose for the pharmaceutically active agent. Preferred doses are in the range of 0.01-10 mM, preferably 0.2-2 mM.

### Compounds

The invention provides a compound of general formula (A):

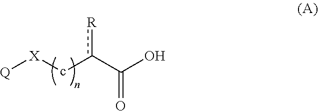

wherein
   X is S, O, Se, or NH;
   c is —CH₂—, —CH(CH₃)—, —C(═O)—, —C(═S)—, —C(═NH)—, —CH(—OH)—, —CH(NH₂)—, —CH(halogen)-, or —C(halogen)₂-;
   n is 1, 2, or 3; and
   R is —H, —CH₃, ═O, ═S, ═NH, —OH, —NH₂, or a halogen; and
   Q is a conjugate of a lipid and a water soluble polymer, wherein the lipid is preferably a phospholipid. Definitions for X, c, n, and R are preferably as described for the ligand earlier herein. General formula (A) without Q can be seen as the ligand. As described above, the means of conjugation is not essential.
Q is a conjugate of a lipid and a water soluble polymer. Preferably, the water soluble polymer is as described elsewhere herein. Preferably the lipid is as described elsewhere herein. Examples of Q are 1,2-distearoyl-sn-glycero-3-phosphoethanolamine-N-[maleimide(polyethylene glycol)-2000], 1,2-distearoyl-sn-glycero-3-phosphoethanolamine-N-[maleimide(polyethylene glycol)-3000], 1,2-distearoyl-sn-glycero-3-phosphoethanolamine-N-[maleimide(polyethylene glycol)-1000], 1-palmitoyl-2-oleoyl-sn-glycero-3-phosphoethanolamine-N-[maleimide(polyethylene glycol)-1000], 1,2-distearoyl-sn-glycero-3-phosphoethanolamine-N-[bromoacetamide(polyethylene glycol)-2000], and 1,2-distearoyl-sn-glycero-3-phosphoethanolamine-N-[acrylamide(polyethylene glycol)-2000].

It is advantageous when the ligand is at one end of the water soluble polymer, and the lipid is at the other end of the water soluble polymer. Examples of compounds of general formula (A) are the conjugates of 1,2-distearoyl-sn-glycero-3-phosphoethanolamine-N-[maleimide(polyethylene glycol)-2000] and mercaptopyruvate or cysteine, preferably L-cysteine, as exemplified in the examples.

### Uses

The invention provides the conjugate according to the invention, for use as a medicament. This encompasses a method of treatment, prevention, or diagnosis of a disorder, wherein the method comprises administering to a subject in need thereof an effective dose of the conjugate. The disorder is preferably a disorder associated with MCT-expressing cells, and can for instance be cancer or a neurodegenerative disorder or a retinal disorder. Preferably the medicament is for treating a neurodegenerative disorder or a retinal disorder, such as inherited retinal degeneration (IRD), glaucoma, age-related macular degeneration (AMD), Stargardt's disease, Usher's disease, geographic atrophy, diabetic retinopathy, retinitis pigmentosa, Leber's congenital amaurosis, blindness, loss of rod photoreceptors, night-blindness, loss of cone photoreceptors, achromatopsia, loss of color vision, and loss of high acuity vision. Preferably the medicament is for treating a neurodegenerative disorder that is also a retinal disorder. Preferred conditions are inherited retinal degeneration, and cone-specific diseases like achromatopsia or age-related macular degeneration, more preferably inherited retinal degeneration. In some embodiments the condition is a cone-specific disease like achromatopsia or age-related macular degeneration.

As is apparent from the Examples, the conjugates for use according to the invention are exceptionally well-suited for delivery of pharmaceutically active agents to the retina. A higher total drug exposure is achieved. This allows the usage of lower doses, which can reduce side effects. IT also allows the effective administration of higher doses, which can improve treatment efficiency, or which can allow less frequent administration to achieve a similar pharmaceutical window or steady state of the drug in the subject.

In a particular embodiment, the delivery of the pharmaceutically active agent is enhanced as compared to the delivery of the same pharmaceutically active agent when it is not present in the conjugate of the invention, e.g. as compared to delivery of a “naked” drug. The conjugates as disclosed herein may be administered daily at least once, twice, three, four, five, six or more times. The lipid nanoparticle or composition as disclosed herein may also be administered once every two, three, four or five days or less often. In preferred embodiments the delivery of an anticancer agent is enhanced as compared to the delivery of the same anticancer agent when it is not present in the conjugate of the invention.

The conjugates of the invention can be said to be photoreceptor-targeted. Utility of photoreceptor-targeted nanoparticles such as liposomes for clinical use can depend on the drug release rate. Rapid drug release can limit the shelf-life of the formulation. For untargeted nanoparticles a low drug release rate can have inferior outcomes, because the concentration of available drug in the target tissue would potentially be too low to achieve an effect. For targeted nanoparticles, conversely, it has been found that lower release rates are beneficial to the therapeutic effect of the drugs, even when there is no difference in the effect between slow- and fast-releasing untargeted liposomes. This can be because the nanoparticles, and thus the drugs, are internalization in target cells, and are thus all released at a relevant location, without systemic scatter. Thus slow release rate can be attractive for photoreceptor-targeted nanoparticles.

Complete release of CN04 from liposomes according to the invention was shown in an in vitro set-up within 48 h. The release rate for CN03 and CN04 can be lowered by precipitation within the liposome cavity with a specific salt using a remote loading technique similar to what has been done for liposomal doxorubicin formulations. CN03 has previously been remote loaded in similar liposomes using calcium acetate salts (Vighi, E., et al., PNAS, 2018, 115(13)).

In some embodiments the conjugate for use is for systemic or intravitreal (IVT) administration. In some embodiments the conjugate for use is for systemic administration. In some embodiments the conjugate for use is for IVT administration. The common routes of drug administration for the treatment of eye disorders are topical, systemic, periocular and intravitreal. Topical administration is preferably topical administration to the eye and can be preferred because of high patient compliance and its non-invasive nature. Upon topical administration, absorption of a drug takes place either through the corneal route (cornea, aqueous humor, intraocular tissues) or noncorneal route (conjunctiva, sclera, choroid/retinal pigment epithelium (RPE)). For naked drugs, only a small fraction of the topically applied drugs, generally less than 5%, reaches the intraocular tissues (Mishra G P et al. J. of Drug Delivery (2011) 2011:863734). Factors responsible for poor ocular bioavailability following topical instillation are precorneal drainage and the lipoidal nature of the corneal epithelium. In addition, a major fraction of the drug reaches the systemic circulation through conjunctival vessels and the nasolacrimal duct, which can lead to adverse effects. Hence, the topical route is not preferred. Systemic administration requires the administration of high doses due to the blood-aqueous barrier and blood-retinal barrier. Such high doses can lead to side effects. For untargeted nanoparticles, intravitreal administration could require frequent administration, which may cause susceptibility for vitreous haemorrhage, retinal detachment and endophthalmitis. Targeted nanoparticles can ameliorate this by effectively delivering higher doses of drugs.

Lactate and MCTs, especially MCT1 and MCT4, are important contributors to tumor aggressiveness (Payen et al., Mol Metab. 2020 March; 33: 48-66). In some embodiments the medicament is for the treatment of cancer. In these embodiments the conjugate preferably further comprises an anticancer agent. Suitable cancers to be treated using the conjugate of the invention are for instance adrenocortical carcinoma, bladder cancer, brain cancer, breast cancer, cervix cancer, colorectal cancer, gastric cancer, head and neck cancer, kidney cancer, liver cancer, lung cancer, lymphoma, ovary cancer, prostate cancer, skin cancer, and soft tissue cancer, and optionally haematological malignancies such as leukemia. Preferred cancers are cervix cancer, lung cancer (particularly NSCLC), lymphoma (such as B-cell lymphoma and Burkitt lymphoma), and skin cancer (particularly squamous cell skin cancer)

### Method

The invention provides a method of treating, delaying, or preventing a neurodegenerative disorder or a retinal disorder, the method comprising the step of administering to a subject a conjugate according to the invention. Features and definition are preferably as defined above.

The invention provides an in vivo, in vitro, or ex vivo method of improving the delivery or potency of a pharmaceutically active agent, the method comprising the steps of:
i) formulating the pharmaceutically active agent in a nanoparticle according to the invention to obtain a nanoparticle comprising the pharmaceutically active agent; and
ii) contacting a cell with the nanoparticle comprising the pharmaceutically active agent. The cell is preferably a cell that expresses MCT, more preferably it is a photoreceptor cell.

### General Definitions

In this document and in its claims, the verb “to comprise” and its conjugations is used in its non-limiting sense to mean that items following the word are included, but items not specifically mentioned are not excluded. In addition the verb “to consist” may be replaced by “to consist essentially of” meaning that a combination or a composition as defined herein may comprise additional component(s) than the ones specifically identified, said additional component(s) not altering the unique characteristic of the invention. In addition, reference to an element by the indefinite article “a” or “an” does not exclude the possibility that more than one of the element is present, unless the context clearly requires that there be one and only one of the elements. The indefinite article “a” or “an” thus usually means “at least one”.

When a structural formula or chemical name is understood by the skilled person to have chiral centers, yet no chirality is indicated, for each chiral center individual reference is made to all three of either the racemic mixture, the pure R enantiomer, and the pure S enantiomer.

Whenever a parameter of a substance is discussed in the context of this invention, it is assumed that unless otherwise specified, the parameter is determined, measured, or manifested under physiological conditions. Physiological conditions are known to a person skilled in the art, and comprise aqueous solvent systems, atmospheric pressure, pH-values between 6 and 8, a temperature ranging from room temperature to about 37° C. (from about 20° C. to about 40° C.), and a suitable concentration of buffer salts or other components.

The use of a substance as a medicament as described in this document can also be interpreted as the use of said substance in the manufacture of a medicament. Similarly, whenever a substance is used for treatment or as a medicament, it can also be used for the manufacture of a medicament for treatment. Products for use as a medicament described herein can be used in methods of treatments, wherein such methods of treatment comprise the administration of the product for use.

In the context of this invention, a decrease or increase of a parameter to be assessed means a change of at least 5% of the value corresponding to that parameter. More preferably, a decrease or increase of the value means a change of at least 10%, even more preferably at least 20%, at least 30%, at least 40%, at least 50%, at least 70%, at least 90%, or 100%. In this latter case, it can be the case that there is no longer a detectable value associated with the parameter. The word “about” or “approximately” when used in association with a numerical value (e.g. about 10) preferably means that the value may be the given value (of 10) more or less 1% of the value.

Each embodiment as identified herein may be combined together unless otherwise indicated. The invention has been described above with reference to a number of embodiments. A skilled person could envision trivial variations for some elements of the embodiments. These are included in the scope of protection as defined in the appended claims. All patent and literature references cited are hereby incorporated by reference in their entirety.

### DESCRIPTION OF THE FIGURES

FIG. 1A—Cellular uptake of ligand-coupled liposomes. Free calcein or liposome-loaded calcein was incubated with HEK293T cells. Shown is the amount of internalized calcein relative to total added calcein determined with fluorescence intensity measurements on a microplate reader. AZD3965 and AR-C155858 are inhibitors of various MCTs. Results presented as mean±SD, *=p≤0.05, ****=p≤0.0001.

FIG. 1B—Cellular uptake of ligand-coupled micelles. Parameters are as for FIG. 1A, except that micelles of either DSPE-mPEG (control) or DSPE-PEG-maleimide-mercaptopyruvate were used, and calcein was replaced by DiO (CAS number 34215-57-1).

FIG. 2A—Distribution of liposome-delivered calcein in organotypic retinal explant cultures. ligand-coupled liposomes (Lp-Pyr and Lp-Cys) containing calcein were added to retinal cultures at post-natal day 15 for 6 h and compared to untargeted liposomes (Lp-OMe), optionally in combination with the MCT1-2 inhibitor AR-C155858. Liposomes were added to the side closest to the ganglion cell layer (GCL). Shown are representative images demonstrating calcein distribution in the retina. IPL=inner plexiform layer, INL=inner nuclear layer, OPL=outer plexiform layer, ONL=outer nuclear layer, Seg.=photoreceptor inner and outer segments. DAPI=nuclear counterstain.

FIG. 2B—As for 2A, shown is calcein signal from each retinal layer. Results represent mean±SD for n=5-6, *=p≤0.05, **=p≤0.01. Statistical analysis: Two-way ANOVA with Tukey's multiple comparison test. Scale bar 50 μm.

FIG. 3A—Treatment of organotypic retinal explant cultures derived from the rd1 mouse model. Retinas were cultured at P5 and treated with either liposome-encapsulated or non-encapsulated (free) neuroprotective drugs (CN03 or CN04) from P7-P11. Assuming an equal distribution across the entire culturing medium, the final drug concentration was 3.14 μM for all treatments. The amount of dying photoreceptors in tissue sections from cultures under various treatment conditions was assessed using the TUNEL assay. DAPI was used to distinguish the outer nuclear layer (ONL). INL=inner nuclear layer. Scale bar 50 μm.

FIG. 3B—As for 3A, shown are percentages of dying (TUNEL+) cells in the ONL. Under non-treated (NT) conditions, the number of TUNEL+ cells are high in the rd1 model. Administration of free CN03 or CN04 had no significant effect on cell death. The same was observed for untargeted, control liposomes (Lp-OMe) and targeted but empty pyruvate-conjugated liposomes (Lp-Pyr). Importantly, Lp-Pyr, when loaded with CN03 or CN04, produced significant photoreceptor protection. Data are presented as mean±SD for n=5-9 animals, ***=p≤0.001, ****=p≤0.0001. Statistical analysis: One-way ANOVA with Tukey's multiple comparison. Scale bar 50 μm.

FIG. 4A—Treatment of organotypic retinal explant cultures derived from the rd10 mice model. Retinas were cultured at P9 and treated from P11 until either P17 or P24 with free neuroprotective CN03 or with CN03 encapsulated in pyruvate-liposomes (Lp-Pyr/CN03). Representative images of retinal explant culture sections at P17 and P24. DAPI used as nuclear stain. ONL=outer nuclear layer, INL=inner nuclear layer.

FIG. 4B—As for 4A, shown is the number of remaining photoreceptor rows in the tissue sections. NT and CN03 substantially overlap. Results represent mean±SD. At P17: n=4-6. At P24: n=4-5. **=p≤0.01 between the Lp-Pyr/CN03 and CN03 or NT group. Statistical analysis: Two-way ANOVA with Tukey's multiple comparison test. Scale bar 50 μm.

### EXAMPLES

### Example 1—Methods

### 1.1 Animals

C3H Pde6b^{rd1/rd1} (rd1), congenic C3H Pde6b^{+/+} wild-type (WT), and C57BL/6J Pde6b^{rd10/rd10} (rd10) mice were housed under standard light conditions, had free access to food and water, and were used irrespective of gender. All procedures were performed in accordance with the association for research in vision and ophthalmology (ARVO) declaration for the use of animals in ophthalmic and vision research and the law on animal protection issued by the German Federal Government (Tierschutzgesetz) and were approved by the institutional animal welfare office of the University of Tübingen. All efforts were made to minimize the number of animals used and their suffering. Animals were not assigned to experimental groups prior to their sacrifice.

### 1.2 Materials

1-palmitoyl-2-oleoyl-sn-glycero-3-phosphocholine (POPC), distearoylphosphatidylcholine (DSPC), 1,2-distearoyl-sn-glycero-3-phosphoethanolamine-N-[methoxy(polyethylene glycol)-2000](mPEG), 1,2-distearoyl-sn-glycero-3-phosphoethanolamine-N-[maleimide(polyethylene glycol)-2000](maleimide-PEG), cholesterol, chloroform (99% with 0.5-1% ethanol), tris(2-carboxyethyl)phosphine (TCEP), thiolactate (95%), L-cysteine, sodium mercaptopyruvate dihydrate, free calcein, hydrogen chloride, sodium hydroxide, disodium hydrogen phosphate dihydrate, sodium dihydrogen phosphate monohydrate, paraformaldehyde, Triton-X (t-octylphenoxypolyethoxyethanol), goat serum, 4-(2-hydroxyethyl)-1-piperazineethanesulfonic acid (HEPES), sodium chloride, proteinase K, fetal bovine serum (FBS), bovine serum albumin (BSA), Corning™ Transwell polycarbonate membranes (0.4 μm, sterile), polycarbonate membranes (0.1 μm pore size), and filter supports were obtained from Sigma-Aldrich (Darmstadt, Germany). Dulbecco's modified eagle medium (DMEM), 1% penicillin/streptomycin, R16 medium and dialysis cassettes (Slide-A-Lyzer, cellulose, 100K molecular weight cut-off) were obtained from Thermo Fisher Scientific (Waltham, MA, USA). The drug compounds CN03 and CN04 were provided by Biolog Life Science Institute (Bremen, Germany).

### 1.3 Preparation of Lipid Nanoparticles

Liposomes with or without conjugation of mono-carboxylate-carrying molecules were prepared using the thin-film rehydration method. Here, a chloroform solution of the lipids POPC, cholesterol, and mPEG were mixed in a molar ratio of 63.3:31.7:5 to produce untargeted liposomes with the end of the PEG chain consisting of a methoxy group (Lp-OMe). Conversely, the lipids POPC, cholesterol, and PEG-maleimide were mixed in the same ratio to produce liposomes with a maleimide group at the end of the PEG chain for subsequent surface conjugation. For CN03 encapsulation, the lipid DSPC was used instead of POPC. All lipid solutions were dried with a rotation evaporator under reduced vacuum (300 mbar) operating at 105 rpm at room temperature. After 1 h, the dried lipids were rehydrated in either of the rehydration buffers listed in table S1 (All mediums adjusted to pH 7.4). For CN03 and CN04 encapsulation, the drugs were added in a 1:3 drug-to-lipid molar ratio. After lipid dissolution in the rehydration medium, 5 freeze-thaw cycles were performed in liquid nitrogen and 37° C. water bath. The liposome solutions were extruded at least 11 times through a PC membrane with 100 nm pores. For conjugation with monocarboxylate molecules, either thiolactate (for lactate-liposomes, Lp-Lac), sodium mercaptopyruvate (for pyruvate-coated liposomes, Lp-Pyr) or L-cysteine (for cysteine-coated liposomes, Lp-Cys) were prepared in 10 mM of the reducing agent tris(2-carboxyethyl)phosphine in 25 mM HEPES (pH 7.4) and added to PEG-maleimide-containing liposomes at twice the maleimide concentration followed by incubation at room temperature for 2 h. To remove non-encapsulated compounds and unbound monocarboxylates, the formulations were dialyzed against isotonic saline at 4° C. For removal of drugs, a 2 h dialysis period was performed. For removal of calcein, a 6 h dialysis period was used with saline exchanged every second hour. The liposomes were sterile filtered and stored at 2-8° C. until further use. The drug concentrations were measured with Ultra High Performance Liquid Chromatography (UPLC) before and after dialysis. The hydrodynamic diameters and ζ-potentials were determined with dynamic light scattering (DLS) (for details see Urimi, D., et al., Int J Pharm, 2021. 602: p. 120640).

TABLE S1
Solutions used for lipid rehydration in liposome preparation.

| | | Liposome |
|---|---|---|
| | Rehydration | concentration |
| Samples | medium | (mg/mL) |
| Calcein-loaded liposomes | 2 mM calcein, PBS | 5 |
| CN03-loaded liposomes | 1.5 mM CN03, 25 mM | 5 |
| | HEPES, 125 mM NaCl | |
| CN04-loaded liposomes | 281 μM CN04, 25 mM | 1 |
| | HEPES, 125 mM NaCl | |
| Empty liposomes | PBS | 1 |
| Liposomes for ζ-potential | 25 mM HEPES | 1 |
| measurements | | |

### 1.4 Uptake of Nanoparticles in Cell Cultures

The human-derived cell line HEK293T were seeded in a 48-well plate at 50,000 cells/well in cell culture medium (DMEM+20% FBS+1% penicillin/streptomycin) for 24 h (37° C., 5% CO₂). Dye-loaded nanoparticles (Lp-OMe, Lp-Lac, Lp-Pyr, or Lp-Cys) were adjusted to 90 μM dye and diluted 1:1 in the medium to give a final concentration of 45 μM. The same concentration of free dye was added. For liposomes calcein was used as dye, for micells DiO (Cas number: 34215-57-1) was used. To determine the role of MCTs in the uptake, some of the cells were pre-treated with either the MCT inhibitors AZD3965 or AR-C155858 at a 2.5 μM and 1 μM concentration, respectively, for 24 h before calcein-loaded Lp-Pyr was added. For control, nothing was added to the cells. After a 2 h incubation period, the wells were washed 3× with preheated PBS. The fluorescent intensities (FI) were measured on a microplate reader (Spark 10M, Tecan, Männedorf, Switzerland) at Ex./Em. wavelengths of 485/530 nm to quantify the amount of intracellular dye. The FI of cells without dye were used as background signal. 90 μM dye in PBS were measured to establish the total FI signal (100%) for quantification. To prepare cells for imaging under fluorescent microscopy, sterile circular glass inserts were added to the wells before the cells were seeding for 24 h. After incubation with nanoparticles, the cells were fixed in 4% paraformaldehyde for 15 min. The glass inserts were transferred to microscopy slides (Superfrost Plus™, R. Langenbrinck, Emmendingen, Germany) and supplied with mounting medium containing DAPI (Vectashield, Vector laboratories, Burlingame, CA, USA). The cells were imaged on a fluorescent microscope (Axio Imager Z2, Zeiss, Oberkochen, Germany) with an ApoTome function with a CCD camera and a 20× objective. A green channel (Ex./Em. 493/517 nm) was used to measure the dye signal, and a blue channel (Ex./Em. 353/465) was used to measure the DAPI signal.

### 1.5 Immunostaining of HEK293T Cells and Murine Retinas

Sterile circular glass inserts were added to a 24-well plate before seeding with HEK293T cells (100,000 cells/well). After 24 h, the cells were fixed in 4% paraformaldehyde and incubated with 0.3% Triton-X in PBS for 5 min at room temperature followed by three times PBS washing. Afterwards, 5% goat serum were added for 1 h. Primary antibodies against MCT1-4 (see table S2) were diluted in 5% goat serum, added to the cells, and incubated overnight at 2-8° C., followed by three times washing. The secondary antibody dissolved in 5% goat serum 1:350 were added and incubated with the cells for 1 h under room temperature and washed.

For immunostaining of murine retinas, wild-type mice were used at post-natal day (P) 30. They were sacrificed by CO₂ asphyxiation and cervical dislocation. The eyes were enucleated and the retinas isolated and fixed in 4% paraformaldehyde, followed by cryoprotection in sucrose as described earlier (Belhadj, S., et al., J. Vis. Exp., 2020). The retinas were submerged in embedding medium (Tissue-Tek O.C.T. Compound, Sakura Finetek Europe, Alphen aan den Rijn, Netherlands), and frozen with liquid N₂. 12 μm thick sections on microscope slides were produced using a cryostat (NX50, ThermoFisher, Waltham, MA, USA). The slides were dried and hydrated with PBS for 10 min. A blocking solution (10% goat serum, 1% BSA, and 0.3% Triton-X in PBS) was added to the slides for 1 h. Primary antibodies against MCT 1-4 and glutamine synthetase (GS) were dissolved in the blocking solution and added to the slides, which were incubated overnight at 2-8° C. Afterwards, they were rinsed with PBS three times, incubated with the secondary antibody for 1 h in the dark, and washed again with PBS. Mounting medium with DAPI was applied, and the slides were imaged using fluorescent microscopy. For detection of MCT1 and GS, the following channel was used: Ex./Em. 557/572. For detection of MCT2, the channel was: Ex./Em. 577/603. For detection of MCT3 and MCT4, the channel was Ex./Em. 493/517.

TABLE S2
List of antibodies used in this study.

| | | Provider, | |
|---|---|---|---|
| Antibody | Type | Cat. no. | Dilution |
| Anti-MCT 1 | Rabbit polyclonal | Alomone Labs, | 1:150 (ICC) |
| | | AMT-011 | 1:100 (IHC) |
| Anti-MCT 2 | Rabbit polyclonal | Alomone Labs, | 1:150 (ICC) |
| | | AMT-012 | 1:100 (IHC) |
| Anti-MCT 3 | Rabbit polyclonal | Abcam, | 1:150 (ICC) |
| | | ab60333 | 1:100 (IHC) |
| Anti-MCT 4 | Rabbit polyclonal | Abcam, | 1:150 (ICC) |
| | | ab180699 | 1:100 (IHC) |
| Anti-Glutamine | Mouse monoclonal | Chemicon, | 1:300 (IHC) |
| Synthetase | | MAB302 | |
| Anti-cone | Rabbit polyclonal | Sigma-Aldrich, | 1:200 (IHC) |
| arrestin | | ab15282 | |

ICC = immunocytochemistry, IHC = immunohistochemistry.

### 1.6 Uptake of Monocarboxylate-Nanoparticles in Organotypic Retinal Explant Cultures

Retinas were isolated and cultured from wild-type mice at post-natal day (P) 13 following a previously established protocol (Belhadj et al., Long-Term, Serum-Free Cultivation of Organotypic Mouse Retina Explants with Intact Retinal Pigment Epithelium. J. Vis. Exp., 2020) and kept in culture until P15, after which a drop of 20 μL 5 mg/mL calcein-loaded liposomes (Lp-OMe, Lp-Pyr, or Lp-Cys) was added to top of the culture on the side corresponding to the vitreoretinal interface. Alternatively, 1 μM AR-C155858 were added to the organ culture medium before addition of Lp-OMe or Lp-Pyr. After a 6 h incubation period (37° C., 5% CO₂), the retinal explant cultures were fixed in 4% paraformaldehyde, cryoprotected in sucrose and frozen with liquid N₂, following the aforementioned protocol. 14 μm sections of retinal explant cultures from the center of the tissue were obtained using a cryostat. The sections were hydrated with PBS for 10 min, supplied with mountain medium with DAPI, and imaged with fluorescent microscopy to record the DAPI signal (Ex./Em. 353/465 nm) and calcein signal (Ex./Em. 493/517 nm). Z-stacks were obtained by recording 11 images 1 μm apart. The stacks were projected using the Maximum Intensity Projection (MIP) function. From these images, the fluorescent intensity was measured, using the acquisition software (ZEN 2.6, Zeiss, Oberkochen, Germany), for each of the following layers: ganglion cell layer, inner plexiform layer, inner nuclear layer, outer plexiform layer, outer nuclear layer, photoreceptor inner and outer segments. Additionally, an immunostaining for cone photoreceptors (cone-arrestin) was performed following the immunostaining procedure for murine retina sections mentioned above on sections from cultures incubated with calcein loaded Lp-Pyr.

### 1.7 Therapeutic Effects of Nanoparticle-Delivered Drugs

Retinas derived from the rd1 or rd10 mouse models were cultured following the protocol described above and treated with the drugs CN03 or CN04. A treatment was done by applying a 20 μL solution containing 160 μM drug (either loaded in liposomes or in a free solution) to the top of the cultures. Assuming an even distribution in the culturing medium, the final drug concentration in the medium would have been 3.14 μM. After fixation with 4% paraformaldehyde, the histological work-up described above was followed to produce 14 μm thick sections. On the rd1 derived culture sections, a terminal deoxynucleotidyl transferase dUTP nick end labeling (TUNEL) assay was performed, detailed under a published protocol (Prajapati, M., et al., Molecules, 2021. 26(5)). Mounting medium with DAPI was applied, and the sections imaged with fluorescent microscopy. For TUNEL detection, Ex./Em. 548/561 nm filters were used. 11 z-stacks 1 μm apart were recorded, and from the projected images, the number of TUNEL-positive cells in the outer nuclear layer was manually counted. For the rd10 derived cultures, the average number of photoreceptor rows residing in the outer nuclear layer was counted from microscopy images, using the same imaging method. Treatment paradigms of organotypic retinal explant cultures derived from either the rd1 or rd10 mouse model were as follows: rd1 treatment started at post-natal day 7 of culturing and stopped at day 11. rd10 treatment started at day 11 of culturing and stopped at day 17 and at day 24. The cultures were treated every second day and stopped by chemical fixation at the indicated time-points. The same paradigm was followed on non-treated cultures.

### Example 2—Results

### 2.1 Expression of MCTs in Retina

We first investigated the expression of MCTs in the retina and specifically on photoreceptors to assess whether these transporters could be used for liposome targeting. Immunostaining for MCT isoforms 1-4 was performed on retinal tissue sections. Different isoforms of MCTs were expressed in different areas of the retina. MCT1 and MCT2 were found to be expressed on photoreceptors. MCT1 was predominately localized to the inner segments of photoreceptors. MCT2 was found on cell bodies in the outer nuclear layer. Due to their localization close to the outer border of the outer nuclear layer, it is likely that these were cone photoreceptors. Most of the cells in the inner nuclear layer also expressed MCT2. MCT3 was not detected in the neuroretina. MCT4 was localized predominately at the vitreoretinal interface. A co-staining with the Müller glial cell marker glutamine synthetase revealed co-expression of MCT4 and the end-feet of Müller glial cells.

### 2.2 Characterization of Monocarboxylate-Nanoparticles

Since MCTs were expressed on retinal photoreceptors, we designed nanoparticle systems for targeting MCTs, for instance a liposome system conjugated with substrates for MCTs. Lactate and pyruvate are known substrates for MCTs and liposomes coupled with these were prepared. Cysteine is structurally similar to both molecules, but is not generally considered a substrate of MCTs, but was also used. All molecules were conjugated to the end of a water soluble polymer, here poly(ethylene glycol) (PEG), linked to the nanoparticle surface. To confirm the successful formation of liposomes, dynamic light scattering (DLS) was performed to measure their hydrodynamic diameter and ζ-potential (Table S3—data represent mean±SD for n=3). The size of conjugated liposomes was the same as that of untargeted, control liposomes. The pyruvate and lactate liposomes showed a more negative surface potential than the control liposomes, indicating a successful conjugation. Since cysteine is neutral at pH 7, the cysteine-liposomes displayed the same ζ-potential as the control.

TABLE S3
physical properties of liposomes

| | Hydrodynamic | | ζ-potential |
|---|---|---|---|
| Formulation | diameter (nm) | PDI | (mV) |
| Untargeted liposomes | 144.4 ± 0.4 | 0.081 ± 0.039 | −9.4 ± 0.5 |
| (Lp-OMe) | | | |
| Lactate-liposomes | 143.9 ± 1.0 | 0.109 ± 0.008 | −28.3 ± 1.7 |
| (Lp-Lac) | | | |
| Pyruvate-liposomes | 143.5 ± 1.3 | 0.108 ± 0.009 | − 28.6 ± 1.9 |
| (Lp-Pyr) | | | |
| Cysteine-liposomes | 145.2 ± 2.5 | 0.117 ± 0.019 | −8.7 ± 0.3 |
| (Lp-Cys) | | | |

### 2.3 Cellular Uptake of Monocarboxylate-Coated Nanoparticles

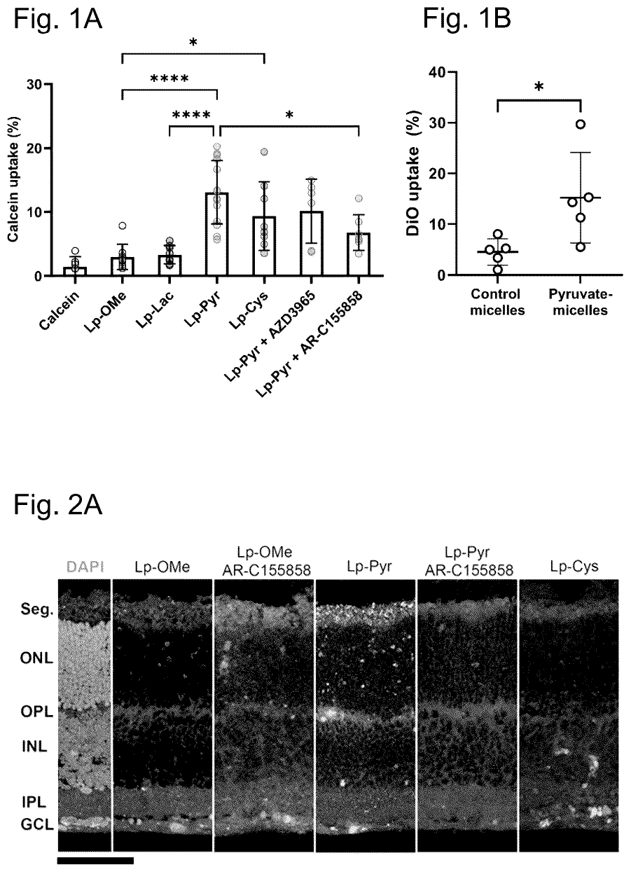

Cellular uptake of monocarboxylate-coated nanoparticles was investigated using a human embryonic kidney cell line (HEK293T). Immunostaining demonstrated the expression of MCT isoforms 1-4 in these cells. Liposomes and micelles were investigated. All liposome formulations (either coupled with monocarboxylate or untargeted) were loaded with calcein, which is a cell impermeable green fluorescent dye, and incubated with the cells for 2 h. Micelles were loaded with DiO (CAS number 34215-57-1). Fluorescent images revealed high uptake of pyruvate- and cysteine-coated liposomes compared to the other conditions. Calcein uptake in the cells was significantly increased with pyruvate- and cysteine-liposomes (FIG. 1A), with the former displaying higher uptake than the latter. Cysteine-coated FePt nanoparticles have previously been shown to be taken up by HEK293 cells (Liang, S., et al., Int J Nanomedicine, 2015. 10: p. 2325-33). Micelles showed comparable behaviour (FIG. 1B).

Interestingly, when inhibitors against MCTs (AZD3965 and AR-C155858) were added, the pyruvate-liposomes delivered less calcein to the cells, demonstrating that the uptake was at least partially mediated by MCTs. When the inhibitor AR-C155858 was used, this difference was significant. Since AZD3965 is selective towards MCT1 (Kᵢ=1.6 nM), while AR-C155858 inhibits both MCT1 and MCT2 (Kᵢ=2.3 and <10 nM, respectively), this suggests that the pyruvate-liposomes are more prone to MCT2-dependent uptake. Even when the AR-C155858 inhibitor was added, there was still a tendency towards higher calcein uptake by Lp-Pyr compared to Lp-OMe. This suggests that in HEK293 cells also MCT3 or MCT4 could mediate uptake. Both of these transporters have been found to take up lactate in vitro.

### 2.4 Retinal Uptake of Pyruvate-Coated Liposomes

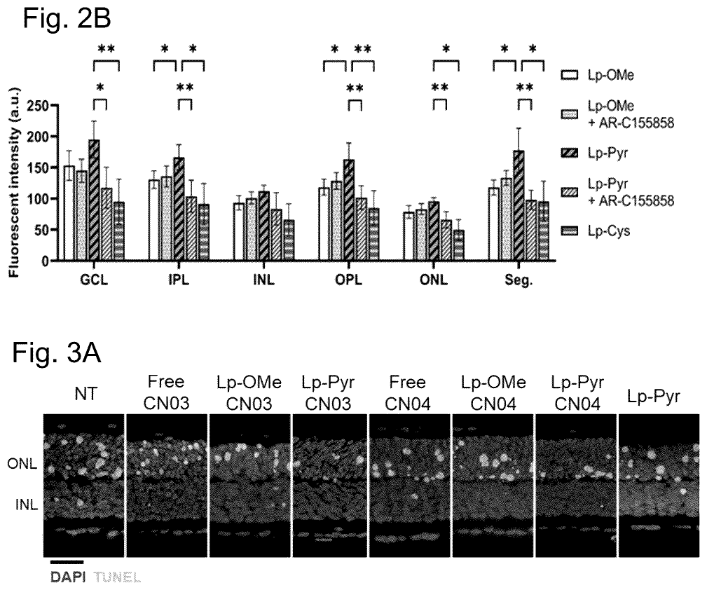

To determine the potential of pyruvate-liposomes to deliver drug to photoreceptors, their uptake in organotypic retinal explant cultures derived from mice were analyzed. Since the target delivery compounds are hydrophilic molecules, a similarly hydrophilic dye, calcein, was used to predict where in the tissue the drugs would accumulate. Calcein was loaded into either the MCT-targeting pyruvate-liposomes (Lp-Pyr), cysteine-liposomes (Lp-Cys), or untargeted control liposomes (Lp-OMe). These were added to retinal explant cultures at post-natal day 15 to the vitreous-facing side of the isolated retinas to simulate the IVT administration. After an incubation period of 6 h, the cultures were fixed, frozen, and sectioned. The amount of calcein dye in the sections was analyzed from fluorescent microscopy images (FIG. 2A) and the signal measured from the distinct retinal layers (FIG. 2B). Lp-Pyr achieved more calcein uptake compared to Lp-OMe in the inner plexiform layer (IPL) and in the outer retina, from the outer plexiform layer (OPL) to the photoreceptor segments. More calcein signal for Lp-Pyr than for Lp-Cys could be detected in the ONL. This comparison is especially relevant since the two formulations share very similar structures and sizes (cf. table S3). The ζ-potential of Lp-Pyr is more negative than Lp-Cys, which normally would be expected to reduce cell uptake. This further demonstrated the ability of the pyruvate-conjugation to drive cell uptake.

To determine whether the higher uptake was mediated by MCTs, the retinal cultures were treated with the MCT1-2 inhibitor AR-C155858 during the incubation period with Lp-Pyr. When the inhibitor was added, an overall decrease in the calcein signal was observed, suggesting that the uptake was mediated by MCTs. Because AR-C155858 is not considered to be an inhibitor for MCT4, our results indicate that MCT1-2 are important for uptake of conjugated particles.

Since MCTs regulate the flow of metabolites in and out of the cells, the inhibition of these key transporters may restrict or slow down the cells' capabilities of taking up liposomes due to low energy conditions or toxic effects. To control for this, Lp-OMe was used in combination with the MCT inhibitor. We found that, unlike the Lp-Pyr uptake, the addition of AR-C155858 did not lead to any reduction of Lp-OMe uptake, suggesting that the transporters themselves are responsible for Lp-Pyr uptake.

From MCT immunostaining results, it appeared that cones predominately expressed MCT2. Since MCT2 is thought to be more specific for pyruvate transport, we tested whether Lp-Pyr was delivered primarily to cones or rods in the outer retina. An immunostaining against the cone specific marker cone-arrestin was performed on sections from cultures to which Lp-Pyr was applied, and the relative amount of calcein-containing rods and cones were determined. We found a tendency to proportionally more cone uptake than rod uptake. Although the difference was not significant (p=0.067), it indicated that Lp-Pyr might be more selective towards cone photoreceptors, a finding that is relevant in the context of cone-specific diseases like achromatopsia or age-related macular degeneration (AMD).

### 2.5 Treatment Effect of Drug-Loaded Pyruvate-Liposomes

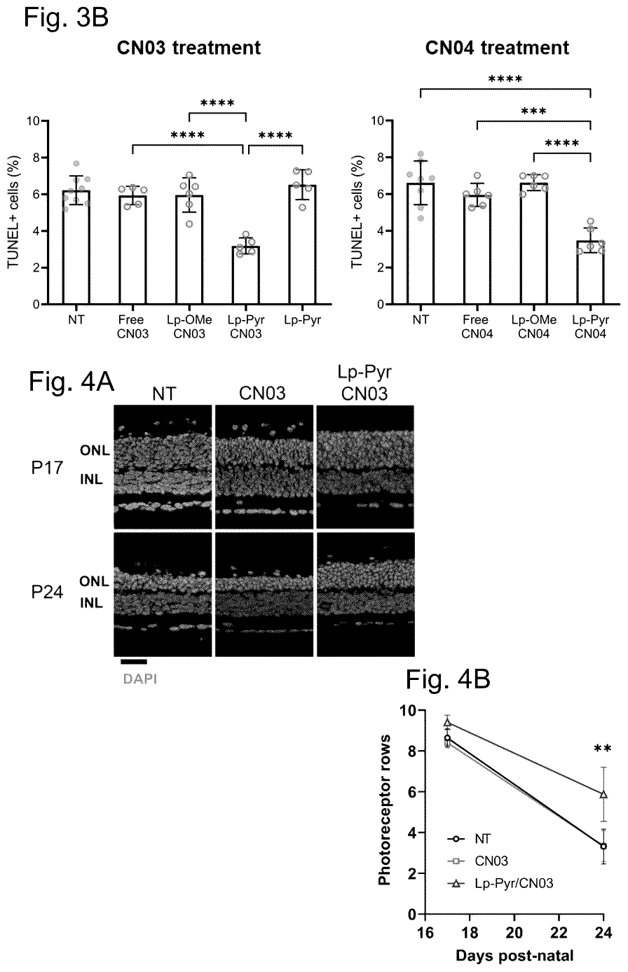

Since the pyruvate-liposomes showed outstanding photoreceptor uptake, we next tested whether these liposomes could enhance the delivery of specific drugs to photoreceptors. Retinal explant cultures derived from the photoreceptor-degeneration mouse model rd1 were used and treated from P7 to P11, i.e., a time-point just before the peak of degeneration. Both free CN03 and CN04, as well as encapsulated compounds were used to assess the effect of the liposome system. The TUNEL assay was employed to detect dying photoreceptors in tissue cross-sections as a read-out of the effect of different treatments (FIG. 3).

When free CN03 or CN04 were applied to the rd1-derived cultures, no significant reduction in dying photoreceptors was obtained, although there was a tendency of reduced cell death. Although CN03 and CN04 are protective in this model, the amount used here (160 μM in 20 μL diluted in 1 ml medium=3.14 μM) was too low to achieve a reduction of cell death when using the free drug solution alone. The Lp-OMe system proved insufficient to transport CN03 or CN04 to photoreceptors. However, the Lp-Pyr system loaded with either CN03 or CN04 could significantly protect the photoreceptors. About 50% less cell death was observed compared to the non-treated control (NT). This protection was significant compared to the free drug solutions and the Lp-OMe system, suggesting that Lp-Pyr actively assisted the transport of drugs to photoreceptors. To make sure that the pyruvate-liposomes themselves were not responsible for the photoreceptor protection, empty Lp-Pyr without drug was tested and found to cause no reduction of dying photoreceptors. The concentration was chosen to be 2 mg/mL as this was the approximate concentration in the Lp-Pyr/CN03 samples. For Lp-Pyr, the encapsulation efficiency was 24.7±6.5% for CN03 and 80.0±5.9% for CN04. Due to the low encapsulation efficiency of CN03, these samples had the highest liposome concentration.

The rate of cell death in the rd1 model is very fast with almost complete rod loss at P18, which is not representative for most IRD patients. A better representation of the human disease situation is afforded by the slower degeneration rd10 mouse model, in which the peak of photoreceptor cell death occurs around P20. Hence, we tested whether pyruvate-liposomes could achieve similar benefits in this model. Here, we tested CN03 encapsulated into liposomes containing DSPC, a formulation that causes slower drug release and is expected to be better suited for in vivo applications. In the rd10 model, we assessed longer-term photoreceptor survival by quantifying the number of photoreceptor rows remaining in the tissue at P17 and P24 (i.e., before and after the peak of degeneration) (FIG. 4). rd10 photoreceptor protection was observed when CN03 was encapsulated in pyruvate-liposomes, while the free drug at the same concentration did not achieve a rescue effect.

### 2.6 Discussion

### 2.6.1 Nanoparticle Uptake in the Retina and Photoreceptors

Efficient retinal drug delivery is a critical concern, notably in the context of IRD. In this study, we have developed a novel approach to deliver cargo to photoreceptors using a targeted nanoparticulate drug delivery system. For instance, we show that pyruvate-conjugated liposomes loaded with neuroprotective drugs achieves efficient photoreceptor protection.

We found that when an MCT-ligand was conjugated to liposome-grafted PEG-chains, more MCT-dependent cell uptake was observed in HEK293T cells and photoreceptor-uptake in retinal explant cultures. Furthermore, this photoreceptor-directed uptake improved the therapeutic effect of photoreceptor rescuing drugs in vitro. We attribute this effect to at least MCT1-2 transporters expressed on photoreceptors. Surprisingly, lactate-nanoparticles did not achieve the same benefit, possibly due to structural differences between lactate and other ligands that were used. This demonstrates that minor differences between the targeting ligand and native substrate can affect transport recognition. This is further exemplified by the fact that our cysteine-liposomes achieved relatively low uptake in the retina despite being structurally similar to pyruvate, and despite showing good uptake in other cell types.

For uptake in the retina, it is likely that the nanoparticles such as liposomes are taken up by Müller glial cells due to the phagocytotic nature of these cells. Trans-retinal permeation of nanoparticles has been documented in the in vivo mouse retina (Lee, J., et al., Mol. Pharm., 2017, 14(2): p. 423-430), which might occur by Müller cell transcytosis and subsequent release in the interphotoreceptor matrix. Some evidence indicates that transporter-targeting can lead to improved transcytosis across tissue barriers such as the blood-brain barrier and the intestinal epithelium. This suggests that for instance pyruvate-liposomes can potentially penetrate the retina better than conventional liposomes, possibly via MCT4-mediated uptake into the Müller cells. Although photoreceptors are not as prone to endocytosis as other cells in the retina, like the Müller cells and RPE, one study found endocytic activity in the inner segments of the photoreceptors (Hollyfield, J. G. and M. E. Rayborn, Exp Eye Res, 1987. 45(5): p. 703-19). Not much information is available about the fate of the transporters following liposome uptake. One study found that the transporters were recycled on the cell surface after the initial liposome uptake, meaning that liposomes did not result in cell-triggered breakdown of the transporters.

We used murine explant retinal cultures, which have several advantages over in vivo models as they are faster, more reliable, and allow studying direct, retina-specific effects. In the explant culture system, we do not preserve the vitreous after the culturing, so the IVT mobility of the nanoparticles was not tested. Previously we have documented the bio-distribution of nanoparticles and found that the retina could be reached after an IVT injection into ex vivo porcine eyes (Eriksen, A. Z., et al., Int J Pharm, 2017. 522(1-2): p. 90-97).

Overall, our study indicates that nanoparticles of the invention are useful as a drug delivery system for the active targeting of photoreceptors.

1. A conjugate comprising:
i) a ligand for a monocarboxylate transporter (MCT); and
ii) a pharmaceutically acceptable nanoparticle.

2. The conjugate according to claim 1, wherein the monocarboxylate transporter is at least monocarboxylate transporter 1 (MCT1), monocarboxylate transporter 2 (MCT2), or monocarboxylate transporter 4 (MCT4).

3. The conjugate according to claim 1, wherein the ligand is a ligand that specifically binds to or is taken up into a retinal cell at a rate that is at least 10% enhanced as compared to control conditions selected from a) uptake in cells lacking expression of monocarboxylate transporter; b) uptake in cells pre-treated with MCT inhibitors; and c) uptake of a reference conjugate lacking a ligand for a monocarboxylate transporter; when measured at 2 hours or more after contacting the ligand with the target cell.

4. The conjugate according to claim 1, wherein the pharmaceutically acceptable nanoparticle is a liposome, a solid lipid nanoparticle, a micelle, a carrier protein, a metal nanoparticle, a polyplex system, a lipoplex system, or a polymeric nanoparticle.

5. The conjugate according to any one of claims 1-4, wherein the pharmaceutically acceptable nanoparticle comprises one or more phospholipids.

6. The conjugate according to claim 1, wherein the pharmaceutically acceptable nanoparticle comprises one or more non-cationic lipids.

7. The conjugate according to claim 1, wherein the pharmaceutically acceptable nanoparticle further comprises a pharmaceutically active agent, preferably a neuroprotective agent, such as a photoreceptor rescuing drug, preferably a cyclic guanosine monophosphate (cGMP) analogue.

8. The conjugate according to claim 1, wherein the ligand comprises a free carboxylic acid moiety, preferably as comprised in a short chain fatty acid, an amino acid, or a keto acid.

9. The conjugate according to claim 1, wherein the pharmaceutically acceptable nanoparticle comprises a water soluble polymer at its surface.

10. The conjugate according to claim 9, wherein the ligand is conjugated to the water soluble polymer.

11. The conjugate according to claim 1, wherein the ligand is of general formula (I): 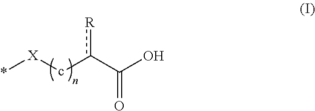
wherein
X is S, O, Se, or NH;
c is —CH₂—, —CH(CH₃)—, —C(═O)—, —C(═S)—, —C(═NH)—, —CH(—OH)—, —CH(NH₂)—, —CH(halogen)-, or —C(halogen)₂-;
n is 1, 2, or 3; and
R is —H, —CH₃, ═O, ═S, ═NH, —OH, —NH₂, or a halogen; and
* is the site of conjugation to the pharmaceutically acceptable nanoparticle.

12. The conjugate according to claim 1, wherein the ligand comprises a free carboxylic acid moiety.

13. A compound of general formula (A): 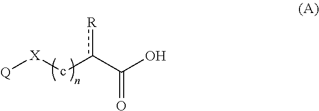
wherein
X is S, O, Se, or NH;
c is —CH₂—, —CH(CH₃)—, —C(═O)—, —C(═S)—, —C(═NH)—, —CH(—OH)—, —CH(NH₂)—, —CH(halogen)-, or —C(halogen)₂-;
n is 1, 2, or 3; and
R is —H, —CH₃, ═O, ═S, ═NH, —OH, —NH₂, or a halogen; and
Q is a conjugate of a lipid and a water soluble polymer, wherein the lipid is preferably a phospholipid.

14.-22. (canceled)

23. Method of treating, delaying, or preventing a neurodegenerative disorder or a retinal disorder, the method comprising the step of administering to a subject a conjugate according to claim 1.

24. The method according to claim 23, wherein the neurodegenerative disorder or a retinal disorder, such as inherited retinal degeneration (IRD), glaucoma, age-related macular degeneration, Stargardt's disease, Usher's disease, geographic atrophy, diabetic retinopathy, retinitis pigmentosa, Leber's congenital amaurosis, blindness, loss of rod photoreceptors, night-blindness, loss of cone photoreceptors, achromatopsia, loss of color vision, and loss of high acuity vision.

25. The method according to claim 23, wherein the conjugate is administered to the subject via topical, periocular, or intravitreal administration.

26. The method according to claim 25, wherein the conjugate is administered to a subject via intravitreal administration.
